(19) United States
(12) Patent Application Publication
(10) Pub. No.: US 2009/0124867 A1
(43) Pub. Date: May 14, 2009

(54) METHOD AND DEVICE TO ADMINISTER ANESTHETIC AND OR VOSACTIVE AGENTS ACCORDING TO NON-INVASIVELY MONITORED CARDIAC AND OR NEUROLOGICAL PARAMETERS

(76) Inventors: Robert A. Hirsh, Merion Station, PA (US); Marc C. Torjman, Southampton, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 11/939,376

(22) Filed: Nov. 13, 2007

(51) Int. Cl. A61M 21/00

(52) U.S. Cl. 600/301; 128/898; 600/509; 600/529; 600/544; 128/203.14; 604/503

(57) ABSTRACT

A method of and a device for non-invasively measuring the neurological depressed state and the hemodynamic state of a human patient and involving steps and units of non-invasively measuring EEG, cardiac cycle period, electrical-mechanical interval, mean arterial pressure, and ejection interval and converting the EEG into a neurological index as well as converting the measured electrical-mechanical interval, mean arterial pressure and ejection interval into the cardiac parameters such as Preload, Afterload and Contractility, which are the common cardiac parameters used by an anesthesiologist. A general anesthetic is administered based upon the converted neurological index. A vasoactive agent is independently administered based upon the converted cardiac parameters as necessary in order to restore cardiovascular homeostasis in the patient. The converted neurological and hemodynamic state of a patient are displayed on a screen as an index value and a three-dimensional vector with each of its three coordinates respectively representing Preload, Afterload and Contractility. Therefore, a medical practitioner looks at the screen and quickly obtains the important and necessary information.

### BACKGROUND OF THE INVENTION

1. Field of the Invention

The present invention relates to a method and device to administer anesthetic agents and vasoactive agents according to non-invasively monitored cardiac and or neurological parameters.

2. Description of the Prior Art

At the present time, since anesthetics or sedative-hypnotic drugs both induce loss of sensation, they are often used for surgical operations. A general anesthetic generally causes a progressive depression of the central nervous system and induces the patient to lose consciousness. In contrast, a local anesthetic affects sensation at the region where it is applied.

Generally, prior to the operation, the patient is usually anesthetized by a specialized medical practitioner (“anesthesiologist”) who administers one or more volatile liquids or gases such as nitrous oxide, halothane, isoflurane, sevoflurane, desflurane, and etc. Alternatively, non-volatile sedative-hypnotic drugs such as pentothal, propofol, and etomidate are administered by injection or intravenous infusion. Opioid analgesics like morphine, fentanyl, or sufenanil are also alternatively administered by injection or infusion, to relieve pain by raising the pain sensation threshold.

In administering a general anesthetic, certain objectives must be properly met for surgery. Firstly, the patient should be sufficiently anesthetized so that his/her movements are blocked. If the patient's movements are not sufficiently blocked, the patient may begin to “twitch” (involuntary muscle reflexes) during the operation, which may move or disturb the operating field that is an area being operated. Such blockage of movement occurs with a depression of the central nervous system after the sensory cortex is suppressed. The depression sequentially affects the basal ganglia, the cerebellum and then the spinal cord. The medulla, which controls respiratory, cardiac and vasomotor centers, is also depressed by the anesthetic in a dose dependent fashion. When respiration is completely depressed by the anesthetic, due to the anesthetic's effect upon the brainstem, it must be performed for the patient by the anesthesiologist, using either a rubber bag or automatic ventilator.

Secondly, the patient should be sufficiently unconscious so as to feel no pain and be unaware of the operation. Patients have sued for medical malpractice because they felt pain during the operation or were aware of the surgical procedure. Once unconsciousness has been achieved, powerful depolarizing and non-depolarizing muscle relaxant drugs can be given to assure a quiescent undisturbed operating field for the surgeon to enhance the first objective. The muscle relaxant drugs, in the unconscious patient allow for a motionless surgical field without the profound central nervous system depression alluded to in to above, which exceeds what is necessary to preclude awareness, and is needed to prevent involuntary unconscious movement in the context of extremely painful stimuli, Using muscle relaxant drugs, which chemically and reversibly disconnect the effect of every voluntary nerve from every voluntary in the body, also increases the risk of intra-operative awareness. This is simply because, a patient with neuromuscular blocking drugs on board, is unable to communicate, verbally or non-verbally, his distress to the anesthesiologist in the event that he should inadvertently emerge into consciousness during the operation. The neuromuscular blocking drugs would prevent only motion, not unconsciousness.

Thirdly, the anesthesia should not be administered in an amount so as to lower blood pressure to the point where blood flow to the brain is reduced to a dangerous extent to cause cerebral ischemia and hypoxia. The dangerous extent is generally below 50 mm Hg for mean arterial pressure (MAP). For example, if the blood pressure is too low for over 10 minutes, the patient may not regain consciousness. This critical pressure will vary with the patient's medical condition. In patients with hypertension, for example, the critical pressure below which injury can occur will be elevated.

A skilled anesthesiologist may monitor the vital signs such as breathing, heart rate and blood pressure of the patient to determine if more or less anesthetic is required. Often, the anesthesiologist looks into the patient's eyes to determine the extent of the dilation of the pupils as an indication of the level or depth of the effect of the anesthesia. The depth is also called “plane of anesthesia.” However, there may be a number of problems with this approach. First, in modem practice, the eyes are frequently taped shut to avoid abrasion or ulceration of the cornea of the eye. Even if the eyes are not covered, judgments about depth of anesthesia depend upon the skill and attention of the anesthesiologist. Some operations may be prolonged for 10 to 15 hours, and the vigilance of the nurse-anesthetist or anesthesiologist may falter or fail. Therefore, it is important to provide a less labor-intensive, yet effective and safe method to monitor and regulate the state of the patient's cardiovascular system.

The state or performance of the cardiovascular system can be described in terms of hemodynamic parameters. One such parameter is the cardiac output (CO). Much effort has been invested in non-invasive methods to measure the CO. (See Klein, G., M.D., Emmerich, M., M.D., Clinical Evaluation of Non-invasive Monitoring Aortic Blood Flow, (ABF) by a Transesophageal Echo-Doppler-Device. Anesthesiology 1998; V89 No. 3A., A953; Wallace, A. W., M.D, Ph.D., et. al., Endotracheal Cardiac Output Monitor, Anesthesiology 2000; 92:178-89). But the cardiac output is just a summary parameter or a final common result of many possible hemodynamic states. In clinical practice, fluid administration and vasoactive drug infusion therapy are not directed to changing the CO per se. Rather, they are directed to the CO's component parameters such as the heart rate (HR) and the Stroke Volume (SV). The relation among the HR, the SV and the CO is given by

CO=HR[SV] Eq. 1

The SV, in turn, is a function of three constituent parameters. The Preload (P) measures the “tension” in cardiovascular muscle at end diastole. The Afterload (A) measures the “resistance” to the blood outflow from the left ventricle. The Contractility (C) measures the rate of rising of the “strain” in cardiovascular muscle. SV increases with increasing P and C and decreases with increasing A. (See Braunwald, E., M.D., ed., Heart Disease, A Textbook of Cardiovascular Medicine, Fourth Edition, Philadelphia, W.B. Saunders Company, 1992, p. 420). In other words, the following relation holds.

SV=f(P,A,C) Eq. 2

where f( ) is a predetermined function.

One way of looking at Eq. 2 is to understand that SV is a function of a vector in a three dimensional space. This vector is just (P,A,C). The axes of the vector space are mutually perpendicular and respectively include P, A, and C. By Eq. 1, CO is linearly proportional to SV by the factor of HR. We can therefore understand that HR is a scalar and operates on a vector in a three dimensional, hemodynamic vector space, H. Substituting Eq. 2 in Eq. 1, we have

CO=HR[f(P,A,C)] Eq. 3

Every possible hemodynamic state in a given system is represented by a unique point in the (P, A, C) space and is scaled by HR. Within H, there is a subset of points that are compatible with life. Furthermore, the subject is defined to be a physiologic hemodynamic vector subspace Ph, which is wholly contained in H. If the position of the hemodynamic vector is tracked in this hemodynamic vector space H by following its trajectory, fairly complete knowledge of the effects of pharmacologic and fluid therapy are available during the perioperative period, Based upon the above knowledge, doctors titrate fluids, diruetics, pressors, afterload reducers, anesthetics, inotropes and negative inotropes against a change in the position of the vector and its relative projection onto each of the three mutually perpendicular axes.

In order to determine the hemodynamic vector, Preload, Afterload, and Contractility as respectively denoted as P, A and C in the above equations must be measured so as to determine Stroke Volume (SV) as defined by Eqs. 2 and 3. Unfortunately, Preload, Afterload, and Contractility have been traditionally assessed by invasive methods.

Preload has been approximated by Pulmonary Capillary Wedge Pressure (PCWP), which is measured with a Swan-Ganz pulmonary artery balloon-tipped catheter that is wedged into the pulmonary arterial circulation. Preload has been approximated by measuring the area of the left ventricle image at end-diastole with 2-D echocardiography.

Afterload has been approximated using the Swan-Ganz catheter to perform thermodilution cardiac output measurements, and measurements of Mean Arterial Pressure (MAP) and Central Venous Pressure (CVP) to calculate the Systemic Vascular Resistance. This is done in analogy with Ohm's law for electrical resistance.

In clinical practice, Contractility is approximated as the cardiac ejection fraction. This requires the methods of nuclear medicine or 2D echocardiography. Alternatively, Contractility is approximated as the maximum rate of rise of left ventricular pressure (LVP) in systole. This is just the maximum value of the first derivative of pressure with respect to time during systolic ejection, or dP/dt max. (See Braunwald, E., M.D, ed., Heart Disease, A Textbook of Cardiovascular Medicine, Fourth Edition, Philadelphia, W.B. Saunders Company, 1992, p. 431). Measuring dP/dt max requires catheterization of the left ventricle. This arrythmogenic procedure is usually reserved for the cardiac catheterization lab since it could be hazardous.

Swan-Ganz catheters are invasive, and their use can be the occasion of clinical mischief. Most experienced clinicians understand the risks associated with using Swan-Ganz catheters in a visceral way. Pulmonary artery rupture, hemo-pneumothorax, pulmonary infarcts, bacterial endocarditis, large vein thrombosis and intraventricular knotting are just a few of the well-known complications that could result from using this device. Some authors have advocated a moratorium on their use, believing that the risks outweigh the benefits. (See Connors, A F Jr., M. D., et. al., The Effectiveness of Right Heart Catherization in the Initial Care of the Critically III Patients, J. Amer. Med. Assn., 1996; 276:889-897; Dalen, J E, Bone R. C.: Is It Time to Pull the Pulmonary Catheter? J. Amer. Med. Assn., 1996, 276:916-8). Although 2-D transesophageal echocardiography devices are prohibitively expensive and also require specialized image interpretation skills, they are still minimally invasive. Because 2-D echo devices require the placement of a large probe in the esophagus, they cannot be used pre-operatively or post-operatively on spontaneously breathing and awake patients. Likewise, the methods of Nuclear Medicine are expensive, requiring a cyclotron to produce specialized radiopharmaceuticals and specialized image interpretation skills. Moreover, since Nuclear Ejection Fractions cannot be done continuously and in real time, they can be used to assess only baseline cardiac function and cannot be used to titrate fluid therapy and drug infusions from moment to moment.

Newer technologies have emerged such as the Hemosonic device from Arrow International (See Klein, G., M.D., Emmerich, M., M.D., Clinical Evaluation of Non-invasive Monitoring Aortic Blood Flow, (ABF) by a Transesophageal Echo-Doppler-Device. Anesthesiology 1998; V89 No. 3A: A953). This minimally invasive device uses a trans-esophageal Doppler placed in the esophagus and one-dimensional A-mode echocardiograph. The Doppler measures velocity of blood in the descending aorta while the A-mode ultrasound is used to measure the descending aortic diameter in real time. Integrating blood velocity times aortic diameter over the ejection interval gives the stroke volume. Stroke Volume times heart rate gives Cardiac Output. Dividing Cardiac Output by the Mean Arterial Pressure gives Systemic Vascular Resistance, Measuring peak blood acceleration gives Contractility. Because the device measures blood flow in the descending aorta, it ignores blood flow to the head and both arms. Thus, it ignores about 30% of the total Cardiac Output and cannot measure Preload. In addition, since the device sits in the thoracic esophagus, it cannot be used on people who are awake.

The above described non-invasive hemodynamic monitoring on a beat-to-beat basis would represent a great improvement in the state of the art, resulting in significant reductions in the cost of care and in perioperative morbidity. Patients are currently monitored invasively. If it were possible to approximate Preload, Afterload, and Contractility using non-invasive means or equipment which is already ubiquitous and relatively inexpensive, it would be a great improvement over Swan-Ganz and Trans-Esophageal Echocardiographic technology. The non-invasive possibility benefits many pediatric patients, renal patients, pregnant patients, and cardiac patients presenting for non-cardiac surgery.

U.S. Pat. No. 7,054,679 has disclosed a low-cost, low risk and non-invasive metric that is used for a wide array of cardiovascular support drug administrations and infusions. Because of its low-cost and low-risk character, a wide range of cardiovascular illness is non-invasively monitored in the operating room and intensive care unit and also from locations outside the traditional hospital settings. It should allow clinicians to pinpoint and quantify the specific causes for acute decompensations in chronic cardiovascular illness and to use this information to modify therapy in such a way as to prevent frequent and costly hospitalization. Accordingly, U.S. Pat. No. 7,054,679 has disclosed apparatuses and methods for continuously and accurately providing real-time information relating to cardiac output in terms of Preload, Afterload and Contractility based upon non-invasive measurements The disclosed non-invasive monitoring technique can track the time evolution of all physiologic compensations prior to a catastrophic decompensation, thereby giving the clinician ample warning and time to intervene beforehand. It also substantially reduces or eliminates the risk of infection arising from the long term use of invasive, indwelling monitoring catheters.

There remains a need for simplifying the administration of anesthetics according to non-invasively monitored cardiac and or neurological parameters. That is, even with the disclosed techniques and devices of U.S. Pat. No. 7,054,679, a competent medical professional must continuously monitor the values of the non-invasively measured cardiac parameters such as Preload, Afterload and Contractility in order to determine a proper response to a change in the hemodynamic state during the course of anesthetic administration. Although the non-invasively measured cardiac parameters accurately reflect the hemodynamic state of a patient, it is still difficult even for a seasoned medical professional to use vasoactive infusion pharmacotherapy to respond in a rational and timely manner to rapidly emerging changes and wide fluctuations in hemodynamic parameters encountered every day in the ordinary practice of anesthesiology. Thus, one major objective of the current invention is to provide a method and an apparatus to process the reliable non-invasively measured cardiac and neurological parameters, and to automatically adjust the administration of anesthetic and vasoactive agents during the course of surgery.

Moreover, it will do so in a way that substantially augments the vigilance and the speediness of timely and appropriate intervention by a competent medical professional. In this way, we can assure the patient's lack of awareness, while insuring a more homeostatic perioperative course, as well as insuring more consistent and more physiologic perfusion of sensitive end-organs such as the brain, heart, kidney, and liver. We conjecture that this improved approach to anesthetic management will decrease morbidity and mortality at one year following anesthesia and surgery (Monk, T. G., e. al., Anesthetic Management and One-Year Mortality After Non-cardiac Surgery, Anesth Analg 2005, 100:4-10).

Peri-operatively, doctors generally use minimally non-invasive cardiovascular monitoring in the current practice to induce anesthesia. Anesthesiologists monitor the heart rate continuously and the blood pressure every five minutes. At intervals, anesthesiologists adjust the volume percent of the inhalational agent or the rate of the Propofol infusion according to the heart rate and blood pressure and administers opioids and muscle relaxants as it seems appropriate.

Unfortunately, this current practice often results in large and frequent deviations from the norms of cardiovascular homeostasis. The anesthetic agent acts both on the brain and the circulatory system to depress the neurological activity and the general circulation. In the current practice, even if the processed EEG shows that the patient is already in a perfectly satisfactory depth of anesthesia, when the patient is hypertensive and hyperdynamic, simply more anesthetic agent is given to the patient. Consequently, the patient may be unnecessarily brought into an even deeper state of neurological depression.

There remains a need for rapidly utilizing vasoactive agents to efficiently restore cardiovascular homeostasis without affecting a depth of anesthesia when the level of neurological depression is appropriate and cardiac parameters indicate abnormal deviations. Afterload-reducing agents include nitroglycerine, nitroprusside, and Nicardipine while Afterload-increasing drugs include phenylephrine. Preload-reducing agents include diuretics and nitroglycerine while Preload-increasing agents include any fluid or plasma expander such as lactated ringers solution, normal saline, 5% human serum albumin, Hetastarch (colloids) and blood or blood products. Contractility-reducing agents include beta-blockers like esmolol while Contractility-enhancing drugs include dobutamine, dopamine, epinephrine and norepinephrine. Non-invasively accessed information contains breath-by-breath changes in the cardiovascular state of the patient, and such changes are indicated by changes in stroke volume, cardiac output, preload, afterload, and or contractility.

There also remains a need for efficiently and safely teaching medical students and inexperienced doctors to administer a general anesthetic. For example, simulation teaches the problem of optimizing fluid administration and the use of diuretics and inotropes (like digitalis and dobutamine) and afterload reducers, like the vasodilator Captopril and its congeners in patients with Congestive Heart Failure (CHF). If a patient has too little fluid, the cardiac output becomes insufficient to perfuse vital organs like the brain, heart, and kidney, resulting in organ failure and death. On the other hand, it a patient has too much fluid, the pumping capacity of the compromised left heart is overwhelmed, allowing fluid to back up into the lungs, causing a diffusion barrier to oxygenation. Fluid welling up in the lungs effectively causes the patient to drown. In this circumstance, patients need to be hospitalized, intubated, and ventilated in an ICU. By adjusting the diuretic dose against the Preload, or its analogue, and by adjusting the Digitalis dose against the contractility, and adjusting the Captopril dose against the SVR or its analogue, patients with CHF are release from the hospital after a shorter period of time.

Lastly, there remains a need for efficiently and safely assaying equipotent doses. For example, equipotent doses are determined for different formulations of the same sedative-hypnotic drugs or inhalational anesthetic agents. Another example of equipotent doses is for different kinds or classes of sedative-hypnotic agents or general anesthetic agents. Since a certain combination of sedative-hypnotic drugs and or general anesthetics may cause non-linear neurological and hemodynamic effects in patients, it is important to monitor the cardiovascular parameters in order to safely reach a desired level of neurological depression. Although there may be some advantage for quickly reaching a certain neurological depression within a shorter amount of time, the non-linear response should be carefully and closely monitored based on the non-invasively measured cardiac parameters on a shorter cycle such as a breath-by-breath basis.

### SUMMARY OF THE INVENTION

One object of the current invention is to permit the clinician to maintain a patient in a particular cardiovascular state and at a certain level of neurological depression with various vasoactive drugs with minimal or substantially no intervention. The cardiovascular homeostasis is automatically maintained with respect to cardiac output, preload, afterload, and contractility.

Another objective of the current invention is to provide a technique and apparatus for simulating a cardio-vascular response in a patient model during anesthetic administration in order to teach inexperienced medical students and develop their skills in anesthesiology.

Yet another objective of the current invention is to assay for equipotent doses of different formulations of the same sedative-hypnotic drugs or inhalational anesthetic agents or equipotent doses of different kinds or classes of sedative-hypnotic agents or general anesthetic agents.

It is to be noted that the scope of this invention is not simply in the sphere of anesthesia, but in the totality of medicine, including outpatient, ambulatory, and critical care medicine.

In a first aspect, the present invention provides a method including steps of non-invasively measuring a set of predetermined non-invasive cardiac and neurological parameters from a subject; converting the non-invasive cardiac parameters into a plurality of invasive cardiac analogues based upon a first set of predetermined conversion equations; administering a general anesthetic based upon the neurological index to maintain a desirable level of neurological depression in the subject; converting the non-invasive neurological parameters into a neurological index based upon a second set of predetermined conversion equations; and independently administering one or more vasoactive agents based upon the converted invasive cardiac analogues to restore cardiovascular homeostasis in the subject.

In a second aspect, the present invention provides a system including a non-invasive neurological parameter measuring unit for non-invasively measuring at least a predetermined neurological parameter from the subject; a neurological parameter conversion unit connected to the non-invasive neurological parameter measuring unit for converting the non-invasive neurological parameter into a neurological index value based upon a first set of predetermined conversion equations; a general anesthetic administering unit connected to the cardiac parameter conversion unit for administering a general anesthetic based upon the neurological index to maintain a desirable level of neurological depression in the subject; a non-invasive cardiac parameter measuring unit for non-invasively measuring a plurality of predetermined non-invasive cardiac parameters from a subject; a cardiac parameter conversion unit connected to the non-invasive cardiac parameter measuring unit for converting the non-invasive cardiac parameters into a plurality of invasive cardiac analogues based upon a second set of predetermined conversion equations; and a vasoactive agent administering unit connected to the cardiac parameter conversion unit for administering a vasoactive agent based upon the invasive cardiac analogues to restore cardiovascular homeostasis in the subject.

### BRIEF DESCRIPTION OF THE DRAWINGS

FIG. 1 is a schematic diagram illustrating major components of a preferred embodiment of the anesthetic and delivering apparatus for administering anesthetic and vasoactive agents based upon non-invasively monitored cardiac and neurological parameters according to the current invention.

FIG. 2 is a schematic diagram illustrating major components of a preferred embodiment of the anesthetic feedback loop in the anesthetic monitoring and delivering apparatus for administering anesthetic based upon non-invasively monitored neurological parameters according to the current invention.

FIG. 3A is a schematic diagram illustrating major components of a preferred embodiment of the vasoactive feedback loop in the anesthetic monitoring and delivering apparatus for administering vasoactive drugs based upon non-invasively monitored cardiac parameters according to the current invention.

FIG. 3B is a diagram further illustrating the cardiac data collection terminal 1006 for non-invasively sampling a patient's cardiac parameters.

FIG. 4 is a schematic diagram illustrating a preferred embodiment of the central monitoring and delivering control unit 1030 according to the current invention.

FIG. 5 is a flow chart illustrating steps involved in a preferred process of administering a general anesthetic agent based upon neurological parameters while maintaining cardiovascular-homeostasis based upon cardiac parameters with minimal human intervention according to the current invention.

FIG. 6 is a flow chart illustrating steps involved in the process of administering a general anesthetic agent based upon neurological parameters with minimal human intervention according to the current invention.

FIG. 7 is a flow chart illustrating further steps involved in the detailed control process of determining a general anesthetic agent dose based upon neurological parameters with minimal human intervention according to the current invention.

FIG. 8 is a flow chart illustration steps involved in the process of administering a vasoactive agent based upon cardiovascular parameters with minimal human intervention according to the current invention.

FIG. 9 is a flow chart illustrating further steps involved in the detailed control process of determining a vasoactive agent dose based upon cardiovascular parameters with minimal human intervention according to the current invention.

FIG. 10 is a diagram illustrating one example of the user interface unit of the present invention.

FIG. 11 is a diagram illustrating one preferred embodiment of the display according to the current invention.

FIG. 12 is a diagram illustrating the user interface unit displaying the deviation of the hemodynamic state vector from a physiological norm as indicative of an amount of physiological stress in one preferred embodiment according to the current invention.

### DETAILED DESCRIPTION OF THE PREFERRED EMBODIMENTS

The detailed description of the current invention incorporates the disclosure from U.S. Pat. No. 7,054,679 by incorporation by external reference.

In general, the current invention enables the clinician to maintain a patient in a particular cardiovascular and neurological state that the clinician judges to be appropriate by conventional means without substantial intervention. By the use of the invention, servo-controlled anesthetic delivery means is controlled to deliver a general anesthetic or sedative-hypnotic drugs in order to maintain a constant level of neurological depression. In addition, servo-controlled infusion means are also controlled to deliver various vasoactive drugs which have substantially no effect on the level of neurological depression in order to maintain cardiovascular homeostasis with respect to Cardiac Output, Preload, Afterload and Contractility. The servo-controlled anesthetic delivery means and the servo-controlled vasoactive drug infusion means are each controlled by a separate and independent feed-back loop. Thus, the current invention substantially functions to provide an ‘autopilot’ or ‘cruise control’ for long anesthetic/surgical procedures. The autopilot mode is safeguarded since the servo-controlled delivery devices are allowed to function within guardrail limits. Should the limits be exceeded, the operator is notified and advised of specifically which physiological parameters require intervention.

The current invention enables the clinician to provide for increased intra-operative vigilance since it is equipped with a 3-dimensional display based upon a predetermined set of algorithms in software. The display indicates three-dimensional vector information on Preload, Afterload and Contractility, myocardial end-diastolic compliance and a linear indicator of level of anesthetic depth. The display also indicates certain conclusion as to whether any intervention is necessary according to the predetermined algorithms. The operator can follow the suggested pharmacological interventions. Optionally, the algorithm also prompts the operator as to when to provide rationally determined interventions in an appropriate time flame. For instance, if the algorithm determines that the cardiac output is falling due to the diminished Preload, it can advise the operator to give a fluid bolus. Then, it subsequently shows the operator just how effective the fluid bolus was in restoring the patient's homeostasis. In addition, the algorithm also includes certain safety features. For example, if predetermined guardrail or safety limitations are approached, the system goes into an open-loop mode. This is analogous to stepping on the brake when a car in cruise control approaches another car in front of it.

The current invention is used to assay for equipotent doses of different formulations of the same sedative-hypnotic drugs or inhalational anesthetic agents. The current invention also determines equipotent doses of different kinds or classes of sedative-hypnotic agents or general anesthetic agents.

The above-described features of the current invention are illustrated in the following drawings to further clarify preferred embodiments. The illustrations are exemplary only and not limiting the current inventions in any manner.

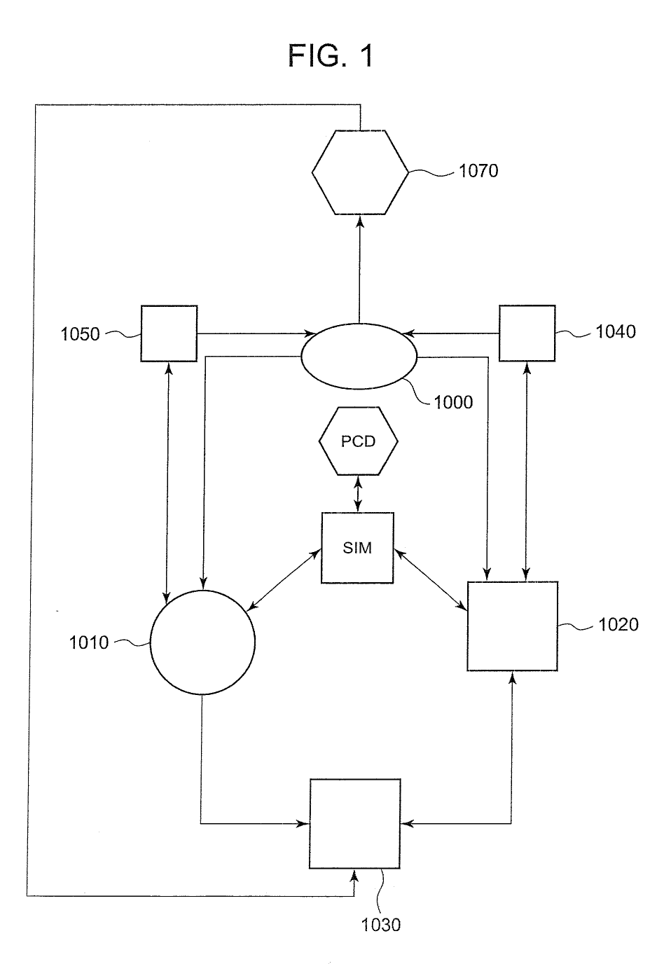

FIG. 1 is a schematic diagram illustrating major components of a preferred embodiment of the monitoring and delivering apparatus for administering anesthetic and vasoactive agents based upon non-invasively monitored cardiac and neurological parameters according to the current invention. One preferred embodiment includes a neurological control unit 1010 and a cardiovascular control unit 1020, both of which are connected to a central monitoring and delivering control unit 1030. The preferred embodiment further includes an anesthetic agent delivery unit 1050, which is connected to the neurological control unit 1010 and a vasoactive agent delivery unit 1040, which is connected to the cardiovascular control unit 1020.

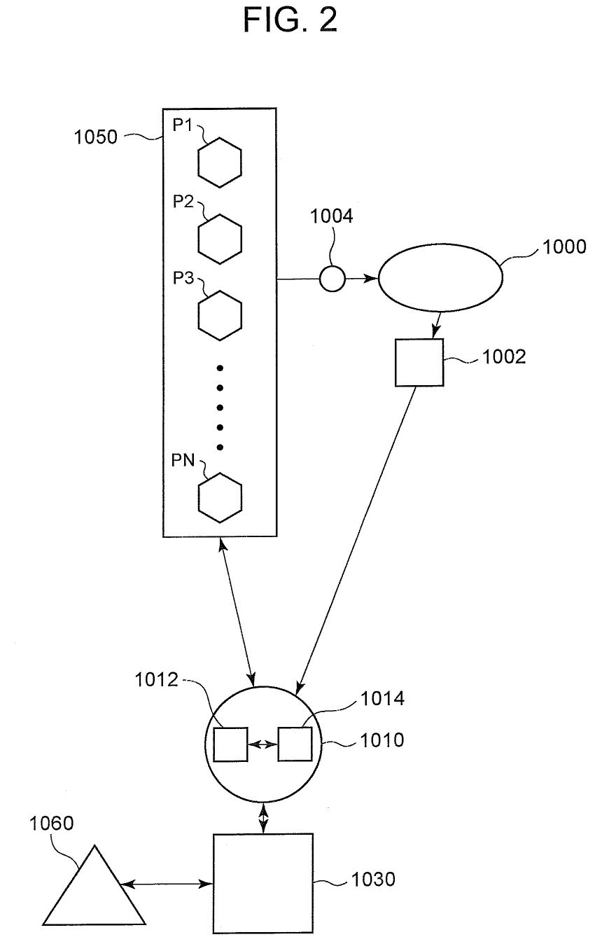

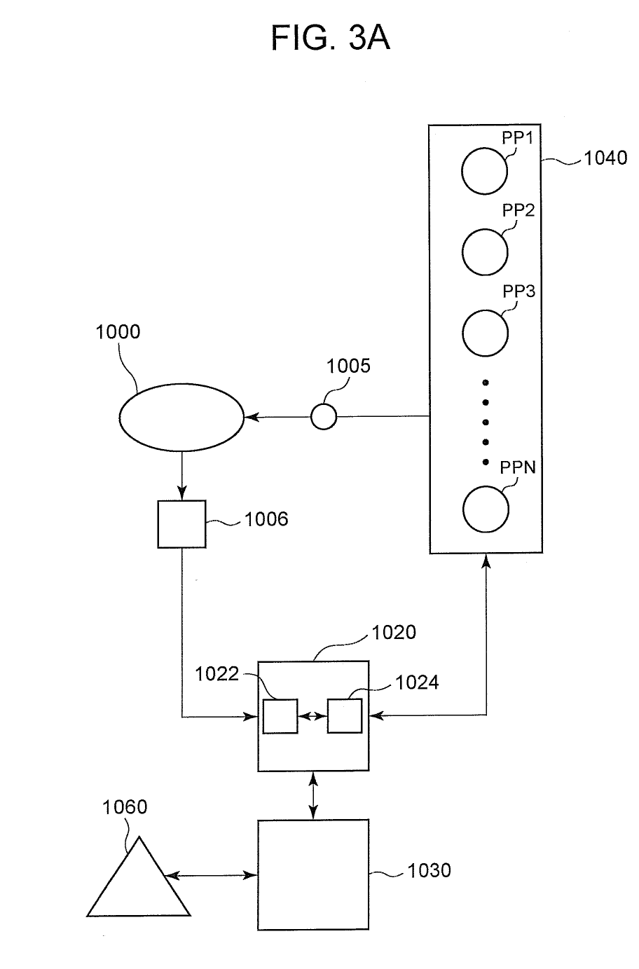

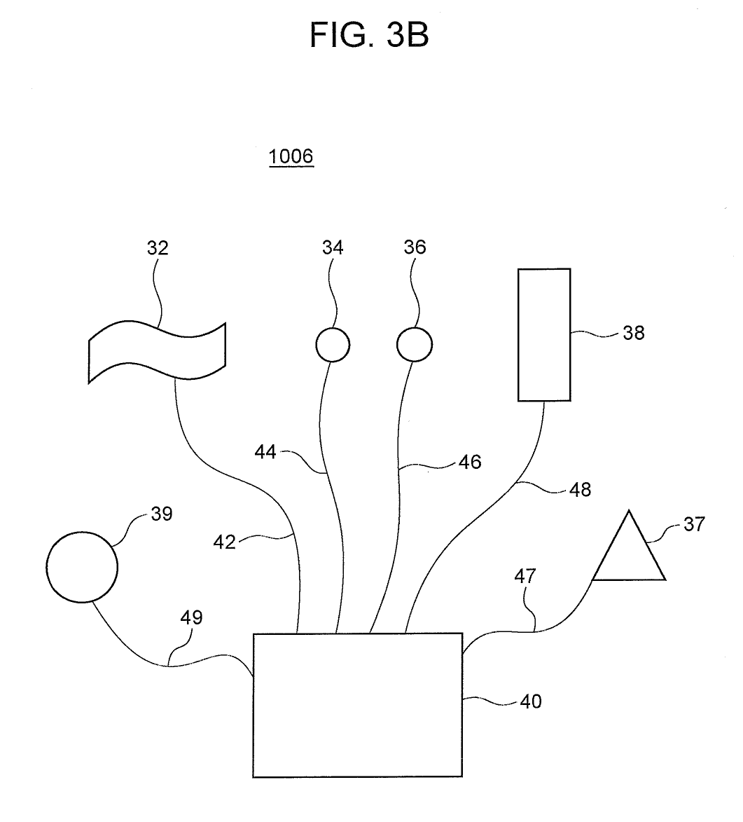

Still referring to FIG. 1, a patient 1000 is connected to two sets of a monitor terminal and an agent delivery terminal. These terminals are not shown in FIG. 1, but will be further described in FIGS. 2 and 3, More specifically, the patient 1000 is connected to the anesthetic agent delivery unit 1050 to receive a proper amount of an anesthetic agent while she is also connected to the neurological control unit 1010 to be monitored for her depth or level of neurological depression. In other words, the neurological control unit 1010 monitors the patient's anesthetic effectiveness and delivers a proper amount of anesthesia based upon the monitored anesthetic depth so as to maintain a desired level of anesthetic effectiveness. At the same time, the patient 1000 is connected to the vasoactive agent delivery unit 1040 to receive a proper amount of a certain vasoactive agent while she is also connected to the cardiovascular control unit 1020 to be monitored for her cardiac parameters. By the same token, the cardiovascular control unit 1020 non-invasively monitors the patient's cardiac parameters and delivers a proper amount of a selective vasoactive agent based upon the monitored cardiac parameters in order to maintain cardiovascular homeostasis within predetermined safe limits.

The two sets of the control unit 1010/1020 and the agent delivery unit 1050/1040 respectively provide an independent feedback loop mechanism for administering anesthetic agents and vasoactive agents according to non-invasively monitored cardiac and neurological parameters according to the current invention. An anaesthetic feedback loop includes the neurological control unit 1010, the anesthetic agent delivery unit 1050 and the central monitoring and delivering control unit 1030 while a cardiovascular feedback loop includes the cardiovascular control unit 1020, the vasoactive agent delivery unit 1040 and the central monitoring and delivering control unit 1030. In general, the two feedback loops are independent of each other since various vasoactive drugs generally have no effect on the level of neurological depression. The central monitoring and delivering control unit 1030 continuously receives data streams from the two independent feedback loops and processes the data streams to generate a separate set of agent delivery commands. In particular, the central monitoring and delivering control unit 1030 generates a set of anesthetic delivery commands to the anesthetic agent delivery unit 1050 based upon the anaesthetic level that is monitored through the neurological control unit 1010. By the same token, the central monitoring and delivering control unit 1030 also generates a set of vasoactive agent delivery commands to the vasoactive agent delivery unit 1040 based upon the cardiac parameters that are monitored through the cardiovascular control unit 1020. These delivery commands include data to specify an agent to be delivered, a pump number, a reservoir number, a rate of delivery and a total amount of the agent.

In an alternative embodiment, the central monitoring and delivering control unit 1030 performs less function than the above described preferred embodiment. Instead of the central monitoring and delivering control unit 1030, the neurological control unit 1010 generates the anesthetic delivery commands to the anesthetic agent delivery unit 1050 based upon the anesthetic level that is monitored through the neurological control unit 1010. Similarly, in an alternative embodiment, instead of the central monitoring and delivering control unit 1030, the cardiovascular control unit 1020 generates a set of vasoactive agent delivery commands to the vasoactive agent delivery unit 1040 based upon the cardiac parameters that are monitored through the cardiovascular control unit 1020.

The above described preferred embodiment in FIG. 1 optionally further includes a third independent feedback loop for monitoring the patient's respiratory-oxygenation condition. A respiratory-oxygenation monitoring unit 1070 in the third optional feedback loop monitors whether or not 1) the patient 1000 is physiologically connected to the ventilator based upon the presence of a CO2 wave form, 2) the CO2 is within physiological limits, 3) a pulse oximeter shows normoxia and not hypoxemia and 4) the in-circuit oxygen sensor shows oxygen in useful concentrations and flows is in fact being delivered to the patient. The respiratory-oxygenation monitoring unit 1070 is operationally connected at least to the central monitoring and delivering control unit 1030 for inputting the above enumerated monitored information to the connected destination units and modules for further processing. Only after the respiratory-oxygenation monitoring unit 1070 has assured that the respiratory and oxygenation parameters are acceptable, the preferred embodiment begins to modulate the cardiovascular-blood flow related parameters using appropriate drug infusions.

The third optional feedback loop substantially helps avoiding the undesirable situation where the reason for ischemia is a lack of oxygen flow into the patient, so that the system does not, for instance respond to an episode of ischemia induced by lack of oxygen flow or respiratory failure by giving more nitroglycerine and or dobutamine, but rather alerting the operator to the fact that the hemodynamic derangement is an undesirable consequence of a respiratory abnormality and failure of oxygen delivery to the lungs.

In yet another alternative embodiment, both the central and local units independently generate a delivery command and the central unit resolves any discrepancy between the two commands. In other words, both the central monitoring and delivering control unit 1030 and the neurological control unit 1010 generate the anesthetic delivery commands to the anesthetic agent delivery unit 1050 based upon the anesthetic level that is monitored through the neurological control unit 1010. When there is any discrepancy between the two commands generated from the central monitoring and delivering control unit 1030 and the neurological control unit 1010, the central monitoring and delivering control unit 1030 resolves the discrepancy. Similarly, both the central monitoring and delivering control unit 1030 and the cardiovascular control unit 1020 generate a set of vasoactive agent delivery commands to the vasoactive agent delivery unit 1040 based upon the cardiac parameters that are monitored through the cardiovascular control unit 1020. By the same token, when there is any discrepancy between the two commands generated from the central monitoring and delivering control unit 1030 and the cardiovascular control unit 1020, the central monitoring and delivering control unit 1030 resolves the discrepancy.

Lastly, the current invention is used as a teaching tool to simulate cardiovascular and neurological conditions of a patient under a general anesthetic. The current invention optionally includes a patient characteristic database PCD and a simulation interface module SIM. The patient characteristic database PCD contains data corresponding to inputs and outputs for the system, and the input and output data represent a predetermined set of patients with a wide range of characteristics such as gender, age, height and weight. The input and output data also includes data representing administration of anesthetic agents and vasoactive agents. In lieu of the patient 1000, the patient characteristic database PCD is connected to the system via the simulation module SIM to initiate, monitor and respond to various conditions that are simulated based upon a particular patient data file in the patient characteristic database PCD. The patient characteristic database PCD and the simulation interface module SIM simulate neurological and cardiovascular conditions including a pulse, an EKG, an EEG, an E-M interval, an EI or DI interval and a blood pressure or a mean arterial pressure of a particular patient. Although the data in the patient characteristic database PCD is generally generated prior to simulation, it is optionally generated during a simulation session in an interactive manner.

The simulation module SIM is connected to the system to utilize available resources in simulating the neurological and cardiovascular conditions of the patient. For example, as shown in FIG. 1, the simulation module SIM is connected to the neurological control unit 1010 and the cardiovascular control unit 1020. The modules and units as will be later described in these control units 1010 and 1020 are efficiently utilized to simulating the conditions in an accurate manner.

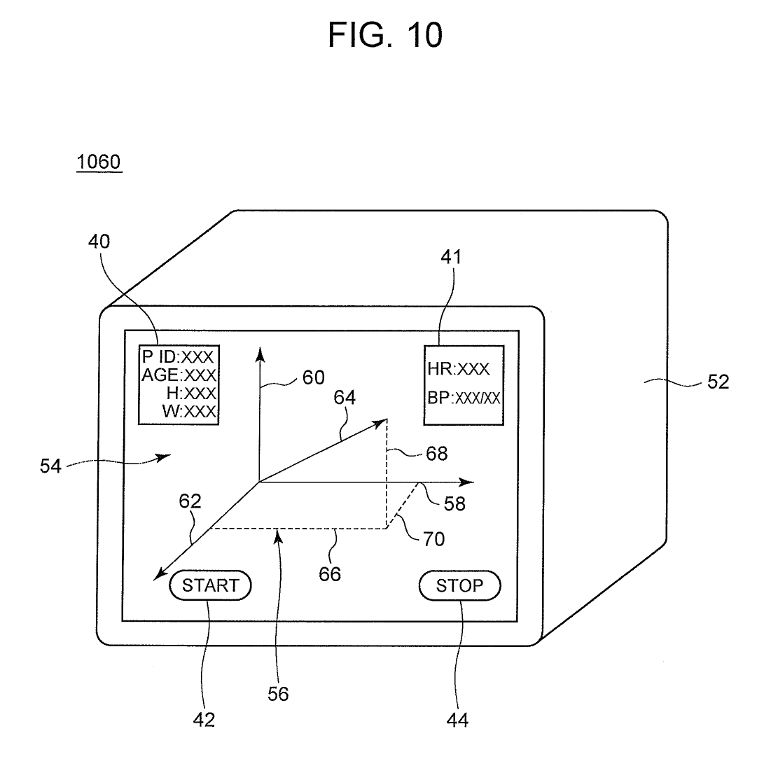

A trainee initiates a general anesthetic with a particular simulated patient and monitors a series of the responses. The trainee understands reasons for an infusion therapy and an anesthetic level adjustment in response to a change in condition via the feedback or instructions as shown on a monitor. The system audio-visually warns the trainee when a dangerous cardiovascular condition exists in the patient. In essence, the purpose of the simulation is to force the trainees to think physiologically about what is happening when it is happening and about what drugs are being infused and why at any particular point in time. The simulation module SIM optionally provides the trainees with description on the above physiological events through a display monitor. Furthermore, a certain simulation mode is optionally selected so that the trainee intervenes with the system and administers a necessary step in a manual mode. The above instructional operations of the system are substantially identical to the user interfaces as will later be described with respect to FIGS. 10 through 12.

FIG. 2 is a schematic diagram illustrating major components of a preferred embodiment of the anesthetic feedback loop in the anesthetic monitoring and delivering apparatus for administering a general anesthetic based upon non-invasively monitored neurological parameters according to the current invention. One preferred embodiment includes a neurological control unit 1010, which is connected to the central monitoring and delivering control unit 1030. The preferred embodiment further includes the anesthetic agent delivery unit 1050, which is connected to the neurological control unit 1010 and the central monitoring and delivering control unit 1030. The patient 1000 is connected to a neurological data collection terminal 1002 for monitoring neurological activities of the brain such as an electroencephalogram (EEG). The patient 1000 is also connected to an anesthetic agent delivery terminal 1004 such as an intravenous catheters, and an inhalation mask, laryngeal mask, or endotracheal tube for delivering a certain anesthetic agent such as a general anesthetic or a sedative hypnotic drug into the patient 1000.

Still referring to FIG. 2, the anesthetic agent delivery unit 1050 further includes a predetermined number of delivery units P1, P2, P3 through PN, each of which is a calibrated and microprocessor-controlled pump or vaporizer for delivering an anesthetic agent. For example, a pump delivery unit P1 delivers a sedative hypnotic agent such as Propofol through an intravenous needle of the anesthetic agent delivery terminal 1004 while a vaporizer delivery unit P2 delivers volatile inhalational anesthetic agents through a breathing circuit connected to a mask, laryngeal mask, or an endotracheal tube. A calibrated volatile agent vaporizer is equipped with a servomotor whose angular displacement adjusts the inhaled concentration of the potent inhalational agent, measured in volumes percent. Optionally, the servomotor attached to the vaporizer is controlled by a microprocessor. Intravenous drug delivery pumps, typically syringe-type pumps such as the ‘Alaris’ pump (Cardinal Health), are servomotor activated, and optionally microprocessor controlled, Each of the delivery units P1, P2, P3 through PN thus further includes at least a reservoir for reserving an anesthetic and a pump with a servomotor to control a pressure level in the reservoir. Each of the delivery units P1, P2, P3 through PN further optionally includes a microprocessor to control the servomotor. After receiving the anesthetic delivery command from either the neurological control unit 1010 or the central monitoring and delivering control unit 1030, the microprocessor controls the rate of delivery by activating the servomotor at a specified rotational speed. The microprocessor subsequently communicates with the neurological control unit 1010 or the central monitoring and delivering control unit 1030 to transmit the information on the delivery such as actual volume and rate of delivery.

The neurological control unit 1010 also further includes a neurological data analysis unit 1012 and an anesthetic level control unit 1014. The neurological data analysis unit 1012 receives neurological brain activity data such as electroencephalogram (EEG) from the neurological data collection terminal 1002. In general, when a patient is awake, the cerebral cortex is active, and the EEG reflects vigorous activities in the brain waveforms. On the other hand, when a patient is sleep or under general anesthesia, the pattern of the brain activity changes to reflect less activity. Based upon a predetermined algorithm, the neurological data analysis unit 1012 analyzes a level of the brain activity to determine a current effectiveness level of a general anesthetic in the patient 1000, who is undergoing surgery. For example, the predetermined algorithm such as Fourier analysis in a computer software program determines whether or not a change from high-frequency signals to low-frequency signals exists due to the anesthetic effect. Another example is that a computer software program determines whether or not there is a tendency for signal correction from different parts of the cortex to become random. In general, the above analyses require a highly computer-intensive process.

One example of a commercially available unit of the neurological data analysis unit 1012 is a bispectral index (BIS) monitor from Aspect Medical Systems. The BIS monitor continually analyses a patient's EEG during general anesthesia to assess the level of consciousness. Raw EEG information is obtained from a patient through a sensor placed on the patient's forehead, and the BIS monitor indicates a single digit ranging from 0 to 100 without any unit. A BIS value of 0 indicates isoelectric EEG or the absence of brain activity while that of 100 indicates fully awake brain activity. The manufacturer recommends a BIS value between 40 and 60 for an ordinary level of general anesthesia. In a preferred embodiment, the BIS value is maintained near 55 or between 50 and 60 Although the BIS monitor has been approved by the Food and Drug Administration (FDA), the exact algorithms used to calculate the BIS index value are not made public. In particular, it measures the distribution of power in Fourier space, the presence of ‘burst suppression’ phenomena in the signal, and also the presence of ‘bicoherence’ between two distinct Fourier components of the same EEG signal, from different frequencies. In the awake brain, the two separate Fourier components stand in little predictable relation to each other. As the brain becomes more progressively depressed, the two separate Fourier components of the EEG become more closely in phase with one another. That is, their ‘bicoherence’ increases. In general, the bispectral index of an EEG is a weighted sum of electroencephalographic subparameters including a time domain, a frequency domain and higher order spectral information (Bispectral Analysis).

Another example of a commercially available unit of the neurological data analysis unit 1012 is ‘Entropy Module’ for its S/5 anesthesia monitoring and delivery system from Datex-Ohmeda, a subsidiary of General EIectric. It uses the ‘Entropy’ algorithm, which yields a first number ranging from 0 to 100 and incorporates the EMG (electromyographic potentials from the frontalis muscle in the forehead) and a second number ranging from 0 to 91. The ‘Entropy’ algorithm also filters the EMG signal. It uses a mathematical definition of entropy and processes the EEG signal according to it. High entropy in the EEG is a sign of wakefulness while low Entropy corresponds to increasing anesthetic depth.

The anesthetic level control unit 1014 is connected to the neurological data analysis unit 1012 to determine as to the use of the analysis output or result from the neurological data analysis unit 1012. In one preferred embodiment, the anesthetic level control unit 1014 generates the anesthetic delivery commands to the anesthetic agent delivery unit 1050 based upon the anesthetic level that is determined by the neurological data analysis unit 1012. For example, the neurological data analysis unit 1012 such as BIS monitor generates a BIS value, and the anesthetic level control unit 1014 processes the BIS value to generate an amount of general anesthetic to be included in the anesthetic agent delivery commands. In another preferred embodiment, the neurological data analysis unit 1012 such as BIS monitor generates a BIS value, and the anesthetic level control unit 1014 compares the BIS value to a predetermined set of threshold values such as the lowest BIS threshold value of 40 and the highest BIS threshold value of 60. If the BIS value goes above 60, there is a statistical chance of awareness. On the other hand, if the BIS value stays low for a long period of time, some clinicians believe that this contributes to increased mortality one year following surgery. Based upon the comparison result, if one of certain situations is identified to require an operator intervention as the anesthetic level is too deep or shallow, the anesthetic level control unit 1014 transmits the BIS value to the central monitoring and delivering control unit 1030 for performing a further clinical analysis and or for taking an additional action such as indicating an operator-warning signal.

Still referring to FIG. 2, the central monitoring and delivering control unit 1030 receives information from the anesthetic level control unit 1014 to further ascertain safety of the patient 1000. It is well known that all of the anesthetic or sedative-hypnotic agents substantially affect the patient's cardiovascular state by virtue of their depressant actions on the autonomic nervous system, myocardial contractility, systemic vascular resistance, preload and cardiac output. Furthermore, manipulations by surgeons also cause the myriad other physiological events during operation. The central monitoring and delivering control unit 1030 takes this information into account to calculate an appropriate dose for delivering the anesthetic or sedative-hypnotic agents to achieve an optimally minimal level of neurological depression so that the patient is safely prevented from experiencing intra-operative awareness as monitored by the processed EEG or a BIS value. In general, it is not necessary or it is even harmful to the patient to be anesthetized to a level beyond the above described minimal neurological depression due to neurotoxicity. The central monitoring and delivering control unit 1030 sends the anesthetic level control unit 1014 the anesthetic delivery commands including the above calculated anesthetic dose data.

The central monitoring and delivering control unit 1030 communicates with a user interface unit 1060, which has an input and or output capabilities to send or receive signals to and from an operator. For example, after it is determined that a warning must be given to an operator for a dangerous anesthetic situation, the central monitoring and delivering control unit 1030 sends an operator-warning signal to the user interface unit 1060 so that the user interface unit 1060 generates an audio warning signal and or a visual warning signal. In response to the operator-warning signal, the operator acknowledges the warning signal or responds to the warning signal through the input device such as a touch screen or a keyboard of the user interface unit 1060. In certain other seriously dangerous situations, the central monitoring and delivering control unit 1030 disengages the anesthetic agent delivery unit 1050 and sends the user interface unit 1060 an operator-warning signal indicating the need for operator intervention. In either case, when the operator enters information via the input device such as a mouse or a keyboard of the user interface unit 1060, the user interface unit 1060 sends the user input data back to the central monitoring and delivering control unit 1030 for further processing. In addition, the user interface unit 1060 continuously displays the updated information on the anesthetic delivery and the anesthetic depth in the patient 1000.

FIG. 3A is a schematic diagram illustrating major components of a preferred embodiment of the vasoactive feedback loop in the anesthetic monitoring and delivering apparatus for administering vasoactive drugs based upon non-invasively monitored cardiac parameters according to the current invention. One preferred embodiment includes the cardiovascular control unit 1020, which is connected to the central monitoring and delivering control unit 1030. The preferred embodiment further includes the vasoactive agent delivery unit 1040, which is connected to cardiovascular control unit 1020 and the central monitoring and delivering control unit 1030. The patient 1000 is connected to a cardiac data collection terminal 1006 for monitoring cardiac parameters of the heart. The patient 1000 is also connected to a vasoactive agent delivery terminal 1005 such as an intravenous catheter for delivering a certain combination of vasoactive agents into the patient 1000. The detailed description of the vasoactive drugs will be given below.

Now referring to FIG. 3B, a diagram further illustrates the cardiac data collection terminal 1006 for non-invasively sampling a patient's cardiac data. The cardiac data collection terminal 1006 includes a cuff 32, two electrodes 34 and 36, an arterial line pressure waveform sensor or a non-invasive T-line 37, a Doppler sensor 38, an EEG sensor 39 and a collection terminal 40. The cuff 32, the electrodes 34 and 36, the non-invasive T-line 37, the Doppler sensor 38 and the EEG sensor 39 are respectively connected to the collection terminal 40 via electrical connections 42, 44, 46, 47, 48 and 49 via common electrical wires. Alternatively, the connections 42, 44, 46, 47, 48 and 49 are wireless connections such as infrared connection and microwave connection that are well known to person skilled in the art. The cardiac data collection terminal 1006 is optionally manufactured to be portable.

The cardiac data collection terminal 1006 is used to measure or monitor the patient. The cuff 32 is attached to the patient's arm or other appropriate body parts for monitoring a heart rate and blood pressure. The sets of the electrodes 34 and 36 are attached to the outer skin in the patient's chest area with a predetermined distance between them for monitoring EKG and heart rate. An additional set of electrodes or a sensor 39 is placed on the head for monitoring EEGs. Furthermore, the Doppler sensor 38 is placed in the suprasternal notch over the ascending aorta or over the carotid artery to measure EI. Alternatively, the Doppler sensor 38 is placed precordially over the left ventricle or in the esophagus of a sleeping patient to measure EI and DI. In addition, the Doppler sensor 38 is optionally replaced by a trans-thoracic cardiac impedance measurement device from the Physioflow Corporation for providing EI, as well as Stroke Volume, and Contractility. The trans-thoracic cardiac impedance measurement device provides the maximum value of the first derivative of impedance with respect to time, dZ/dt as contractility. The cardiac data collection terminal 1006 optionally controls the frequency of data acquisition from the patient and outputs the collected data to the cardiovascular control unit 1020.

Referring back to FIG. 3A, the vasoactive agent delivery unit 1040 further includes a predetermined number of delivery units PP1, PP2, PP3 through PPN, each of which is a calibrated and microprocessor controlled infusion pump or flow regulator for delivering a vasoactive agent. The vasoactive agent can be an intravenous liquid or an inhalational gas such as Nitric Oxide (NO), which is used in the treatment of critically ill people with pulmonary hypertension. For example, a first pump delivery unit PP1 delivers a vasoactive agent such as dobutamine or nitroglycerine while a second pump delivery unit PP2 delivers another vasoactive agent such as esmolol or phenylephrine through an intravenous catheter of the vasoactive agent delivery terminal 1005. Each of the delivery units PP1, PP2, PP3 through PPN thus further includes at least a reservoir for reserving a vasoactive agent and a pump with a servomotor to control a pressure level in the reservoir. Each of the delivery units PP1, PP2, PP3 through PPN further optionally includes a microprocessor to control the servomotor.

After receiving the vasoactive agent delivery commands from either the cardiovascular control unit 1020 or the central monitoring and delivering control unit 1030, the microprocessor controls the rate of delivery by activating the servomotor at a specified rotational speed for a specified time interval. The microprocessor subsequently communicates with the cardiovascular control unit 1020 or the central monitoring and delivering control unit 1030 to transmit the information on the delivery such as actual volume and rate of delivery.

The cardiovascular control unit 1020 also further includes a cardiac data analysis unit 1022 and a cardiac parameter control unit 1024. The cardiac data analysis unit 1022 receives in real-time non-invasively measured cardiac or hemodynamic data from the cardiac data collection terminal 1006 and generates cardiac parameter data such as Cardiac Output, Preload, Afterload and Contractility. According to a predetermined algorithm, the cardiac data analysis unit 1022 monitors cardiovascular homeostasis of the patient 1000 in terms of the cardiac parameters. The generation process of the cardiac parameters will be further described in detail later.

The cardiac data analysis unit 1022 is implemented based upon the following cardiac parameters and relationships among them. Left Ventricular End-Diastolic Pressure (LVEDP), Systemic Vascular Resistance (SVR) and the Maximum Rate of Rise of Left Ventricular Pressure (dP/dtmax) are respectively clinically useful indices and invasive cardiac analogues of or approximations to Preload, Afterload and Contractility, (P,A,C). Even though these respective pairs of cardiac parameters are not perfectly linear with respect to one another, they are monotonically increasing with respect to each other. Therefore, LVEDP, SVR and dP/dmax are also cardiac parameters that are responsive to cardiac medicines such as fluids and diruetics, pressor's and afterload reducers, anesthetics, inotropes and negative inotropes. That is precisely why clinicians can rely on LVEDP, SVR and dP/dmax to administer the proper dosage of medicines for further controlling these parameters and therefore for adjusting the state of hemodynamics of the patient.

In addition, it is an accepted tenet of physiology that a complete description of the functional state of the heart is given by four parameters. They are the heart rate, the LVEDP, the SVR, and dP/dmax. (See Braunwald, E., M.D., ed., Heart Disease, A Textbook of Cardiovascular Medicine, Fourth Edition, Philadelphia, W. B. Saunders Company, 1992, p. 374-82). The last three of these, which determine the stroke volume, has been typically obtained only at the cost of invasion of the patient.

To non-invasively measure cardiac parameters, the following relationships have been already described in the description of the prior art and will be used:

SV=f(P,A,C) Eq. 2

where f( ) is a predetermined function, and SV is Stroke Volume.

CO=HR[f(P,A,C)] Eq. 3

where CO is Cardiac Output and HR is Heart Rate.
By making the appropriate substitutions for Preload, Afterload, and Contractility, we can rewrite Eqs. 2 and 3 respectively as:

SV=f(LVEDP,SVR,dP/dtmax) Eq. 4

CO=HR[f(LVEDP,SVR,dP/dtmax)] Eq. 5

Therefore, the state of a hemodynamic system is substantially described based upon the above four parameters. Three of these parameters constitute a vector in a three-dimensional vector space, H′. The axes of H′ are LVEDP, SVR, and dP/dt max with appropriate units. A function ‘f’ , of this vector determines the stroke volume, SV. The fourth parameter, the heart rate HR operates linearly as a scalar on the vector to determine the cardiac output, CO.

The cardiac data analysis unit 1022 converts a second plurality of cardiac parameters into a first plurality of cardiac parameters that are directly responsive to external medicines. In a preferred embodiment of the present invention, the first plurality of cardiac parameters are LVEDP, SVR, and dP/dtmax, which are directly responsive to cardiac medicines such as fluids and diruetics, pressors and afterload reducers, anesthetics, inotropes and negative inotropes. More preferably, the first plurality of cardiac parameters further includes heartrate (HR). The second plurality of cardiac parameters is non-invasively measured directly using proper instrumentation including mean arterial pressure (MAP), the Ejection Interval (EI), the Diastolic Filling Interval DI, and Electrical-Mechanical Interval (E-M). More preferably, the second plurality of non-invasively measured parameters further includes Heart Rate (HR), which together with MAP, EI and E-M substantially gives a complete description of the function state of the heart.

EI is the time interval during which systolic ejection takes place. It starts when the aortic valve opens and ends when it closes. If an ordinary Doppler ultrasound device is placed over the suprasternal notch near the ascending aorta, inspection of the frequency vs. time curve will yield the EI. Also, since the time from mitral valve closure to aortic valve opening in systole is small compared to the Ejection Interval, the interval from the first heart sound to the second heart sound measured using a stethoscope or phonocardiogram is optionally a useful approximation to the EI.

E-M is defined by the time between two specific events, an electrical event and a mechanical event. The electrical event is an event detectable on the EKG, which initiates ventricular contraction. The electrical event can be the Q-wave, the R-wave or the S-wave. In each case, Q, R or S is respectively defined as a point in time when the Q-wave, the R-wave or the S-wave reaches a particular point such as a maximum, a minimum or other predetermined point on the wave. The event is optionally a ventricular pacing spike.

In some arrhythmias, like ventricular tachycardia with a pulse, there IS no Q-wave (or R-wave, or S-wave). Therefore, another embodiment of ‘E’ of the E-M interval is to look at the EKG waveform defining ventricular depolarization, differentiate it twice with respect to time, and define the point in time at which the electrical depolarization wave accelerates maximally upward as ‘E’. This would allow for the definition of an E-M interval in those instances where there is no recognizable Q, R, or S wave, i.e. when the patient is in extremis. For instance, in ventricular tachycardia, the waveform looks like a rapid sine wave. This alternative embodiment of ‘E’ may also turn out to be a practically more accurate way to determine E-M by more accurately defining ‘E’, to within narrower tolerances. The main point is to find and accurately define a physiologically identical time point in all possible EKG ventricular depolarization cycles, which are then compared to one another to create consistent usable E-M intervals.

The mechanical event is a palpable consequence of ventricular contraction. It is related to the electrical event and lags the electrical event in time. The upstroke of the arterial trace from an indwelling arterial catheter qualifies as a mechanical event, and so does the instant at which the upward acceleration of the arterial pressure trace is at maximum. In other words, a mechanical event occurs at the instant of maximum value of the second derivative of pressure with respect to time. If the arterial blood pressure (ABP) is given by A(t), then the mechanical event is given by A″(t) max or A″max for simplicity, Therefore, in one embodiment, the E-M interval (E-M) is further defined as Q-A″max, R-A″max or S-A″max.

If we place a Doppler device over a major artery such as the ascending thoracic aorta near the sternal notch, then the instant of flow velocity upstroke with the onset of systole qualifies as a mechanical event. If Doppler detected flow is given by F(t), then the instant at which the acceleration in flow is maximum or F″(t) max is also, a useful mechanical event. Therefore, in another embodiment, the E-M interval (E-M) is defined as Q-F″(t) max, R-F″(t) max or S-F″(t) max.

A useful mechanical event is also obtained from the upstroke of the optical plethysmographic curve using a pulse oximeter placed on a patient's finger, toe, nose or earlobe. Similarly, the instant of maximum upward acceleration of the plethysmographic curve (PM(t)) is a clinically useful mechanical event. In one embodiment, the mechanical event is defined as the instant at which the PM(t) curve hits a minimum prior to the detection of flow. Alternatively, the mechanical event is defined as the instant at which PM(t) curve accelerates maximally upward as flows become rapid. Differentiating the PM(t) curve twice with respect to time give us the PM″(t). The instant at which PM″(t) reaches a maximum value, following the Q-wave (or its substitutes) defines a Q-PM″(t) max interval, which is a further embodiment of the E-M interval (E-M).

The onset of the first heart sound, representing the closure of the mitral valve optionally likewise serves as a useful mechanical event. The instant of maximum amplitude of the first heart sound is optionally used as a mechanical event as well. It matters little which event is used to define the E-M interval according to the current invention. By analogy, the E-M interval is like the interval between a flash of lightning and a clap of thunder. It matters only that we use the same one consistently when making comparative judgments.

A particular mechanical event is detected using a physiologic sensor developed at Empirical Technologies Corp to define the E-M interval. This technology uses a fiberoptic device that sits over the radial artery and vibrates with the arrival of the pulse wave. The vibration of the fiberoptic element due to the arterial pulse wave affects the transmission of a beam of light inside.

Another embodiment detects the mechanical event by placing a fiberoptical seismometer device over a large artery to measure the displacement of the arterial wall transverse to the direction of blood flow. The displacement of the arterial wall transverse to the direction of blood flow with respect to time t is defined as TD(t). By analogy, an E-M interval is defined as Q-TD″(t) max. TD″(t) max is the time when TD″(t), which is the second derivative of TD(t), reaches its maximum value.

Using the interval between the trough of the Q-wave on EKG and the upstroke of the arterial pressure wave in a major artery, the Q-A interval (one type of E-M interval), the quantification of myocardial Contractility was first described in a letter to the editor of the Lancet by Jackson, in 1974. (See Jackson, D. M., M.D, A Simple Non-Invasive Technique for Measuring Cardiac Contractility, [Letter]. Lancet 1974; ii:1457). Using human volunteers, he plotted the decrease in the Q-A interval from baseline at one-minute intervals, while infusing isoproteranol. As the infusion came to equilibrium, he described a linear decrease in the Q-A interval with respect to time. He then doubled the rate of the infusion and obtained a further linear decrease in the Q-A interval over time. Of interest, at the lower rate of isoproteranol infusion, the Q-A interval significantly decreased in comparison to baseline while the heart rate changed relatively little, This showed that the decreased Q-A interval was due to an increase in the inotropic state of the myocardium and not due to an increase in the heartrate. He also described a positive correlation between dP/dtmax and the decrease in the Q-A interval in anesthetized beagle dogs with left ventricular catheters. He affirmed this correlation using five different agents, all of which have an effect on the inotropic state of the myocardium, thiopental, calcium, isoproteranol, norepineprine and digitalis.

In another letter to the editor of the Lancet two months later, Rodbard (See Rodbard, S., Measuring Cardiac Contractility, [Letter]. Lancet 1975; I: 406-7) indicated that he had used Jackson's approach for at least a decade earlier particularly in the diagnosis and evaluation of hyperthyroid and hypothyroid states. Rodbard described the measurement of the interval from the Q-wave to the Korotkoff sound over a major artery, the Q-Korotkoff interval (Q-K interval) as well as using a Doppler ultrasound device placed over a major artery to generate a Doppler frequency shift versus time curve (D(t)) to measure the Q-D(t) interval (or Q-D interval).

In contrast, according to the present invention, a more preferred mechanical event is defined by D″(t) max, the time t at which D″(t) reaches maximum value following the peak of the Q-wave or its substitute. Similarly, D″(t) is derived by differentiating D(t) twice against time t.

In general, by differentiating a physiologic function M such as A(t), PM(t), F(t), TD(t) or D(t) twice to obtain the time of a useful mechanical event, an improved accuracy of E-M is achieved. In a preferred embodiment, the mechanical event of E-M is defined as E-M″max, where M″max is defined at the time when M″, which is obtained by double differentiating the physiologic function M against time t, reaches a particular maximum.

The shorter the E-M interval is, the greater the Contractility of the myocardium becomes. The relation between Q-A, Q-K or Q-D interval and Contractility or dP/dtmax has long been in the public domain. (See Cambridge, D., Whiting, M., Evaluation of the Q-A interval as an Index of Cardiac Contractility in Anesthetized Dogs: Responses to Changes in Cardiac Loading and Heart Rate. Cardiovascular Research 1986; 20: 444-450). However, as will be disclosed later, the E-M interval is not only correlated with Contractility but also is used to correlate with other cardiac parameters which are responsive to medicines.

Empirically, cardiac output and hemodynamic state of a patient are correlated to HR, EI, MAP and E-M, which are the second plurality of cardiac parameters that are non-invasively measured in a direct manner. By equating the right hand members of eqs. 2 and 4, the following equations are provided:

(P,A,C)=(LVEDP,SVR,dP/dtmax) Eq. 6

where both sides of the equations can be thought of as a vector in a 3-dimensional Cartesian Space. We can show that

(LVEDP,SVR,dP/dtmax)=f(EI,MAP,E-M) Eq. 7

where f is a mathematical transformation that changes the orientation and length of the vector. The above relations are mathematically and logically equivalent to the relations among the invasively measured quantities (P, A, C) or its equivalents (LVEDP, SVR, dP/dtmax).

A first three-dimensional non-invasive vector space M with three mutually perpendicular axes EI, MAP and E-M is constructed even though each of these three axes does not change linearly with Preload, Afterload, or Contractility. In particular, Preload does not vary linearly with EI, Afterload does not vary linearly with MAP, and Contractility does not vary linearly with E-M. Nor does each of these three axes vary directly in any useful or predictable way with the infusion of a particular class of vasoactive medications. For every point in the invasive hemodynamic vector space H′, there exists exactly one corresponding point in the non-invasive hemodynamic vector space M. Moreover, every point in the non-invasive hemodynamic vector space M has an image in the invasive hemodynamic vector space H′. In the language of linear algebra, there is a mathematical mapping from the non-invasive hemodynamic vector space M to the invasive hemodynamic vector space H′ in a ‘one-to-one’ and corresponding manner. Therefore, in one aspect, the present invention demonstrates there is a one-to-one correlation between the non-invasive hemodynamic vector space M and the invasive hemodynamic vector space H′.

A particular hemodynamic state vector in the (EI, MAP, E-M) space does not directly show the equivalents or analogues of the invasive parameters such as (P, A, C) or (LVEDP, SVR, dP/dtmax). In order to get to an analogue vector in the (P, A, C) or (LVEDP, SVR, dP/dtmax) space from the non-invasively measured vector in the (EI, MAP, E-M) space, a predetermined transformation on the (EI, MAP, E-M) vector is needed. Therefore, the cardiac data analysis unit 1022 converts between the above described two vectors in the (EI, MAP, E-M) space into an equivalent vector in the (P, A, C) or (LVEDP, SVR, dP/dtmax) space. This conversion may be implemented in many different forms such as a computer program residing on a computer.

The cardiac data analysis unit 1022 transforms by multiplying the (EI, MAP, E-M) vector by a diagonal matrix as shown below. Let x be a vector in the non-invasive hemodynamic space M of the form (EI, MAP, E-M) Let A be the diagonal matrix shown below. If we represent x vertically as a column vector, we can multiply it by the matrix A such that Ax=b, where b is a vector of the form ((EI*MAP*E-M), (MAP*E-M), 1/(E-M)), that is approximately equivalent to (LVEDP, SVR, dP/dtmax), and a first embodiment of the first plurality of cardiac parameters responsive to external medicines as being demonstrated in the equation below.

$$( \begin{matrix}{MAP}^{*} ( E \text{-} M ) & 0 & 0 \\ 0 & E \text{-} M & 0 \\ 0 & 0 & 1 / {( E - m )}^{2}\end{matrix} ) ( \begin{matrix}EI \\ MAP \\ ( E \text{-} M )\end{matrix} ) = ( {EI}^{*} {MAP}^{*} ( E \text{-} M ) , {MAP}^{*} ( E \text{-} M ) , 1 / ( E \text{-} M ) )$$

The above operation of multiplying the vector by a matrix linearly transforms the vector x into the vector b. Vector b constitutes a new vector space N or a second Non-invasive Space whose axes are responsive to external medicines and fluid administration as being verified below. The three mutually perpendicular axes of the vector space N are EI*MAP*E-M, MAP*E-M, and 1/E-M. The first axis, (EI*MAP*E-M) is linearly proportional to the LVEDP to a first approximation. The second axis, (MAP*E-M) is linearly proportional to SVR to a first approximation. The third axis, (1/E-M) is linearly proportional to the natural logarithm of dP/dtmax or ln(dP/dtmax) to a first approximation. These relations are summarized as follows:

LVEDP=k1(EI*MAP*E-M)+c1 Eq.8

SVR=k2(MAP*E-M)+c2 Eq.9

ln(dP/dt)max=k3(1/E-M)+c3 Eq.10

Solving Eq. 10 for dP/dt max,

dP/dtmax=Z[exp(k3/E-M)], where Z=exp(c3) Eq. 11

where k1, k2, k3, and c1, c2, c3 are empirical proportionality constants.

Eqs. 8 through 11 are true only to a first approximation. That is because while the left hand members of Eqs. 8 through 10 do increase monotonically with respect to the right hand members, the increases may not be perfectly linear with respect to one-another. As the patient deviates further from the physiologic norm, the size of the non-linearity increases. This is because the relations between the left and right hand members of Eqs. 8 through 10 are more subtly exponential than linear. So within an arbitrarily large neighborhood of a given physiologic point, the tangent to the subtle exponential curve gives a reasonably good approximation. However, since they are monotonically increasing with respect to one-another, they are practically useful in controlling proper medicine administration. (EI*MAP*E-M), (MAP*E-M) and (1/E-M) are used to judge the changes in the Preload, Afterload, and Contractility due to fluid and drug administration.

In addition to generating a data stream of hemodynamic state vectors describing Preload, Afterload, and Contractility on a beat-to-beat basis, the cardiac data analysis unit 1022 also yields a similar data stream about Stroke Volume, SV. A truly remarkable and useful property of the vector space M is that SV is a function of only two of the non-invasive quantities, the Ejection Interval (EI) and the E-M interval (E-M). In other words, the following equation expresses the relation

SV=f(EI, E-M) Eq. 12

Let the average rate of outflow of blood from the Left Ventricle during the ejection interval be Fei in cc/sec. Then by definition, the following relation exists.

Fei=SV/EI Eq. 13

Based on the experimental results disclosed in the present invention, Fei is empirically and linearly proportional to the transcendental number e^{1/E-M}. The quantity 1/E-M is the time rate at which electromechanical transduction and elastic propagation of the pulse wave or analogous mechanical events occur. So we can write,

Fei=k4*exp(1/E-M)+c4 Eq.14

where k4 and c4 are empirical proportionality constants. Solving Eq. 13 for SV, we have

SV=EI*Fei Eq. 15

Substituting for Fei using Eq. 14, Eq. 15 becomes either:

SV=EI*[k4*exp(1/E-M)+c4] Eq.16

SV αEI*[exp(1/E-M)] Eq. 16a

Where “α” means “proportional to.” There are alternative formulations of SV such as the length or norm of the vector sum of two orthogonal vectors. One of the two orthogonal vectors is a function of EI, and the other is a function of (E-M).

Alternatively, the cardiac data analysis unit 1022 uses the diastolic filling interval (DI) to replace EI in eq. 8, which tracks Preload as LVEDP. The correlation is improved between (DI, EI, MAP, E-M), which is of the second plurality of non-invasively measured cardiac parameters and (LVEDP, SVR, dP/dtmax) or (P, A, C), which is the first plurality of invasive cardiac parameters in a second embodiment. In diastole, the left ventricular pressure is an exponential function of left ventricular volume, and this relation holds at any point during the diastolic filling interval including end-diastole. Therefore, LVEDP is an exponential function of Left Ventricular End Diastolic Volume (LVEDV). Physiologically, it makes intuitive sense that, other things being equal, the longer the length of time that the Left Ventricle fills in diastole, DI, the more volume of blood will fill the left ventricle at end-diastole with a higher resulting LVEDP, or Preload. To a reasonable approximation,

DI=T−EI Eq. 17

where T is the time period of the cardiac cycle. T is easily obtained in a non-invasive manner by measuring the time interval between R-waves in the EKG and is linearly proportional to the reciprocal of the heart rate, HR in beats per minute. That is,

T=(1/HR)*60 sec/min Eq. 18

The above approximation ignores the time required for isovolumic contraction and relaxation. However, since the two intervals are relatively small fractions of any cardiac cycle, the approximation is useful.

A more accurate measure of DI is optionally obtained using a 1 MHz Doppler ultrasound device placed on the surface of the patient's chest just over the left ventricle. Alternatively, a Doppler device can be placed in the retro-cardiac esophagus in a patient under anesthesia. Diastolic filling has a characteristic low velocity blood flow that causes an analogously low Doppler frequency shift. The duration of the characteristic low frequency Doppler shift substantially serves as an accurate measure of DI. DI starts when the mitral valve opens, and it ends when the mitral valve slams shut. An ordinary stethoscope or phonocardiogram generally indicates when DI ends as marked by the first heart sound, the ‘lub’ of the two sounds ‘lub-dub’. In patients with certain pathology, an opening snap of the mitral valve is audible in the stethoscope. Perhaps a phonocardiogram shows when the mitral valve opens in most patients. Alternatively, the above described fiberoptic sensor that is placed upon the precordium of the chest serves as a low cost ‘seismometer’ to measure the duration of the low frequency vibrations by diastolic filing in the amplitude of the fiberoptic light signal. The Doppler device is more expensive but has the advantage for obese patients. Therefore, the correlation between (DI, MAP, E-M) and (LVEDP, SVR, dP/dtmax) or (P, A, C) is defined by the following equations in the preferred embodiment:

LVEDP=k1′((T-EI)*MAP*E-M)+c1′ Eq. 19

SVR=k2′(MAP*E-M)+c2′ Eq. 20

ln(dP/dt)max=k3′(1/E-M)+e3′ Eq. 21

where k1′, k2′, k3′, c1′, c2′ and c3′ are constant for a particular patient.

Other hemodynamic parameters being equal, the longer the time interval over which the left ventricle fills, the higher its end-diastolic volume and pressure becomes. That is, the longer DI is, the higher the LVEDP becomes. If the EI by itself varies in a useful way with LVEDP, this is due to a law of physiology relating EI to DI in the steady state. E1 by itself has no primary causal relation to LVEDP since it is defined by two events that occur in the cardiac cycle after the left ventricle has finished filling. The quantity DI=T-EI is logically, temporally and physiologically prior to the LVEDP. EI by itself is logically, temporally and physiologically posterior to LVEDP.

Using the above described correlation, The cardiac data analysis unit 1022 provides real-time and non-invasive measures to be used to express Preload, Afterload, Contractility, Stroke Volume, Heartrate, Cardiac Output, and Average Ejection Outflow Rate. From the foregoing equations, it is relatively simple to derive useful expressions for Left Ventricular Ejection Fraction, whose units are dimensionless, Left Ventricular Stroke Work in units of Joules, and Left Ventricular Power in Watts.

The existence of the correlation between the first plurality of cardiac parameters and the second plurality of cardiac parameters is verified by using the method of converting the second plurality of non-invasive cardiac parameters into the first plurality of cardiac parameters that are measured independently and invasively. The methods of measuring the first plurality of non-invasive cardiac parameters are well known to person skilled in the art. The following are exemplary methods.

Using the averaged waveforms, LVEDP is obtained by inspecting of the LVP(t) waveform and looking for the value of LVEDP just prior to the rapid increase in LVP due to systole. Contractility is obtained by differentiating the LVP(t) curve with respect to time, and recording the maximum value of the first derivative during systolic ejection, dP/dtmax. Afterload, which is approximated by Systemic Vascular Resistance (SVR) is obtained by the known formula (See Kaplan, J. A., M.D., Cardiac Anesthesia, Philadelphia, W. B. Saunders Company, 1993, p. 63)

$$SVR = \frac{( MAP - CVP ) * 80}{CO} \qquad Eq . 22$$

where MAP is the Mean Arterial Pressure in mmHg, and CVP is the Central Venous Pressure in mmHg, CO is the cardiac output in liters/minute. It is obtained using the thermodilution technique, with a Swan-Ganz catheter thermistor connected to a digital temperature vs. time curve integrator. The constant having a value of 80 is used to convert mmHg/(liter/min) into dyne*sec*cm⁻⁵. CVP was recorded by hand from the monitor at each steady state as with MAP. HR, the heart rate/min, is obtained by measuring the period of the averaged EKG, taking its reciprocal and then multiplying by 60 sec/min. The HR is divided into CO to get the Stroke Volume (SV).

To create the non-invasive hemodynamic state vectors, the following approach is used. The Diastolic Filling Interval, (DI) is just the time from the opening of the mitral valve until it closes. The Ejection Interval EI is just the time from the opening of the aortic valve until it closes. These measurements are easily obtained using a precordial or retro-cardiac esophageal Doppler ultrasound device. When the valves open, the Doppler measured blood velocity rapidly increases. When the Valves close, the Doppler measured blood velocity becomes zero. The interval from end of a zero velocity state to beginning of the next zero velocity state just prior to the EKG QRS complex is the DI. The interval from the end of the zero velocity state at the time of the EKG QRS complex to beginning of the last zero velocity state is exactly the ejection interval, EI. Mean Arterial Pressure (MAP) is simply read from the monitor display. Alternatively, the ABP waveform is integrated over the cardiac period, and then the integral is divided by the period to get MAP, denoted MAPc. It does not make a significant difference which approach was used. EI is also easily obtained with an acoustic Doppler device placed in the suprasternal notch over the ascending aorta. MAP is easily obtained using a blood pressure cuff and a DINAMAP. These devices are ubiquitous and relatively inexpensive. Alternatively, MAP may be obtained non-invasively using a T-line, manufactured by Tensysmedical.

In yet another alternative approach to non-invasive hemodynamic parameter measurement, it is possible to dispense with the need for measurement of the E-M interval, a metric of contractility, provided that another non-invasive technology is used to measure the Stroke Volume (SV), SV can be measured non-invasively using several new technologies. One example is the Hemosonic 100, by Arrow International, which uses ultrasound to measure descending aortic blood flow and diameter and computes SV by integrating flow times aortic cross sectional area over the EI. Another example by USCOM makes the SV measurement and an EI measurement without trespassing the esophagus by putting the transducer in the suprasternal notch. A third example by the Physioflow measures SV and EI on the basis of trans-thoracic impedance measurements. A fourth example by Linton uses Pulse Contour Analysis of the radial arterial pulse wave to determine stroke volume on a beat-to-beat basis. This can be combined with a precordial or suprastemal Doppler ultrasound device to measure EI, Using these devices, it is simple to calculate the quantity, SV/EI or the average rate of blood outflow through the aortic valve during systole.

In a related patent application (Ser. No. 11/689,934) filed on Mar. 22, 2007, by the present inventor, a relation between SV/EI and dP/dtmax was disclosed.

ln(SV/EI)=A+B*ln(dP/dtmax) eq. 23

where A and B are empirically determined constants. But by eq, 10, ln(dP/dtmax) α(1/(E-M)) This means that

ln(SV/EI)α1/(E-M) eq. 24

Solving eqs, 10 and 24 for (E-M), we have

(E-M)α1/[ln(dP/dtmax)] eq. 25

(E-M)α1/[ln(SV/EI)] eq. 26

It follows that in any of the forgoing mathematical transformations of a vector in a non-invasive vector space to a vector in an invasive vector space, that it is possible to substitute 1/[ln(SV/EI)] for (E-M) and still preserve the linearity of the transformation equations. Mathematical equality is easily obtained from linear proportionality simply by adjusting the constant A and the coefficient B of the proportion for the change in units. This enables us to construct a noninvasive vector space which spans and reflects events in the invasive vector space out of the basis vectors {HR, EI, DI, MAP, SV}. We can designate this alternative non-invasive Vector Space N′. Moreover, given a stream of noninvasive hemodynamic data, as described above, it enables us to provide a mathematically and physiologically complete description of the hemodynamic state of the cardiovascular system. More importantly, as regards the present invention, it is possible to use that information to inform the decision of what vasoactive medications to use, and in what dose, at what time, and for how long in a servomechanically directed computer-controlled feedback loop. This is done to maintain perfusion homeostasis in the face of a level of neurological depression which is judged to be adequate to assure unconsciousness. In a 49 year old male volunteer undergoing rigorous exercise testing, the quantity SV/EJ, measured with a Physioflow trans-thoracic impedance measuring device, ranged from 250 cc/sec at rest to 1200 cc/sec at maximal exercise. The physiologic range pf patients with medical conditions such as Congestive Heart Failure, Coronary Artery Disease or severe Aortic Valve Stenosis, will be significantly less.

The cardiac parameter control unit 1024 is connected to cardiac data analysis unit 1022 to determine as to the use of the cardiac parameter data from the cardiac data analysis unit 1022. In one preferred embodiment, the cardiac parameter control unit 1024 generates the vasoactive agent delivery command to the vasoactive agent delivery unit 1040 based upon the cardiovascular homeostatic level in terms of the cardiac parameters that are determined by the cardiac data analysis unit 1022. According to predetermined algorithms, the cardiac parameter control unit 1024 analyzes cardiovascular homeostasis of the patient 1000 based upon the cardiac parameter data values. For example, the predetermined algorithm continuously attempts to restore cardiovascular homeostasis in response to the cardiovascular mischief caused by an anesthetic agent and surgical manipulation so that all vital organs are properly perfused and oxygenated. In this regard, a computer software program determines parameter specific vasoactive substance infusion therapy on a breath-by-breath basis to compensate for the cardiovascular imbalance in order to restore cardiovascular homeostasis.

The parameter specific vasoactive substance infusion therapy generally means a calculated amount of a certain vasoactive agent to be infused for restoring cardiovascular homeostasis. The calculated amount and the vasoactive agent are specified in the vasoactive agent delivery command. Although the above analyses require a highly computer-intensive process, the substantially real-time infusion therapy will likely reduce postoperative complications such as one-year mortality. In another preferred embodiment, the cardiac data analysis unit 1022 generates a set of the cardiac parameter values, and the cardiac parameter control unit 1024 compares the cardiac parameter values to a predetermined set of threshold values. In one preferred embodiment, the threshold values are set with respect to a normal state of an individual patient before anesthesia, which is represented as a vector in {EI, DI, MAP, E-M, Heartrate} as will be later described in detail. The respective threshold values are set to +20% or −20% of what is normal for a particular patient. In practice, it is a simple matter to measure the parameters {EI, DI, MAP, E-M, Heartrate} (vector space M) in the conscious and unstressed patient while the patient is exercising on a treadmill according to the standard Bruce protocol. If the patient cannot exercise, a standard Dobutamine Stress test, using a standard Dobutamine infusion protocol, such as is regularly used in radionuclide cardiac imaging and ejection fraction measurement is employed. The percentage thresholds for each parameter in M could be set in a customized, tailored fashion, based upon what the patient is known to tolerate from the exercise testing setting. For example, based upon the comparison result, if one of many possible critical situations is identified to require an operator intervention as the Preload value is too high or low, the cardiac parameter control unit 1024 transmits the Preload value to the central monitoring and delivering control unit 1030 for performing a further clinical analysis and or takes an additional action such as indicating an operator-warning signal.

In the absence of predetermined threshold settings for the parameters in vector space M for a particular patient, certain reasonable default settings can be used. Alternatively, the thresholds are determined based upon individual profile, exercise stress or dobutamine infusion stress test. Reasonable limits on heart are from 60 to 100 beats per minute. Reasonable limits on MAP are 60 to 115 mmHg For hypertensive patients, the lower limit is higher. Based on the Weissler Regression Formula for the Left Ventricular Ejection Time indexed to heartrate, LVETi=LVET+1.65(HR), (Ref. Weissler, A. M., Harris W. S., Shoenfeld C. D., Circulation (1968, 37; 149-159), and given the physiologic range of heart rate, then EI (which is identical to LVET) will range from 235-360 milliseconds in an average, healthy 40 year old man. From this, it follows that the diastolic filling interval DI should range from 305-700 milliseconds. Assume now that E in the E-M interval is defined as the point in time of maximal upward acceleration of the EKG depolarization voltage during the QRS complex, and M is defined as the point in time of maximal acceleration in the concomitant pulse wave transduced at the right wrist using a non-invasive T-line. Measurements made on human volunteers undergoing exercise stress testing show that, for a 49 year old 5′10″ male in good health, E-M will range from 186-106 milliseconds. Bear in mind that the shorter the value of E-M, the more contractile and hyperdynamic the heart. Setting limitations on thresholds for the parameters in M will require the use of sound clinical judgment, even as that is now required in judging the parameters commonly used in clinical practice.

Still referring to FIG. 3A, the central monitoring and delivering control unit 1030 receives information from the cardiac parameter control unit 1024 to further ascertain safety of the patient 1000. It is well known that all of the anesthetic or sedative-hypnotic agents substantially affect the patient's cardiovascular state by virtue of their depressant actions on the autonomic nervous system, myocardial contractility, systemic vascular resistance, preload and cardiac output. Furthermore, manipulations by surgeons also cause the myriad other physiological events during operation. The central monitoring and delivering control unit 1030 takes these information into account to calculate an optimal and safe dose for delivering the vasoactive agents. The central monitoring and delivering control unit 1030 sends the cardiac parameter control unit 1024 the vasoactive agent delivery commands including the optimally safe dosage information.

The central monitoring and delivering control unit 1030 communicates with the user interface unit 1060, which has an input and or output capabilities to send or receive signals to and from an operator. For example, after it is determined that a warning must be given to an operator for a dangerous cardiovascular situation, the central monitoring and delivering control unit 1030 sends an operator-warning signal to the user interface unit 1060 so that the user interface unit 1060 generates an audio and or visual warning signal. In response to the operator-warning signal, the operator acknowledges the warning signal or responds to the warning signal through the input device such as a touch screen or a keyboard of the user interface unit 1060. In certain other seriously dangerous situations, the central monitoring and delivering control unit 1030 disengages the vasoactive agent delivery unit 1040 and sends the user interface unit 1060 an operator-warning signal indicating the need for operator intervention. In either case, when the operator enters information via the input device such as a mouse or a keyboard of the user interface unit 1060, the user interface unit 1060 sends the user input data back to the central monitoring and delivering control unit 1030 for further processing. In addition, the user interface unit 1060 continuously displays the updated information on the cardiovascular homeostasis in terms of the cardiac parameters in the patient 1000.

Furthermore, in certain exemplary applications of the current system according to the current invention, the cardiac data collection terminal 1006 of the present invention is placed on a patient at home and is made to communicate via the Internet to a website from which his or her physician downloads the patient's hemodynamic data prior to surgery. Furthermore, the cardiac data collection terminal 1006 is optionally made small enough to be worn by the patient for collecting pre-operative hemodynamic data of a patient. The monitored information is stored in the wearable unit for over an extended period beyond 24 hours. The cardiac data collection terminal 1006 is alternatively used in the management of outpatients with high blood pressure or congestive heart failure.

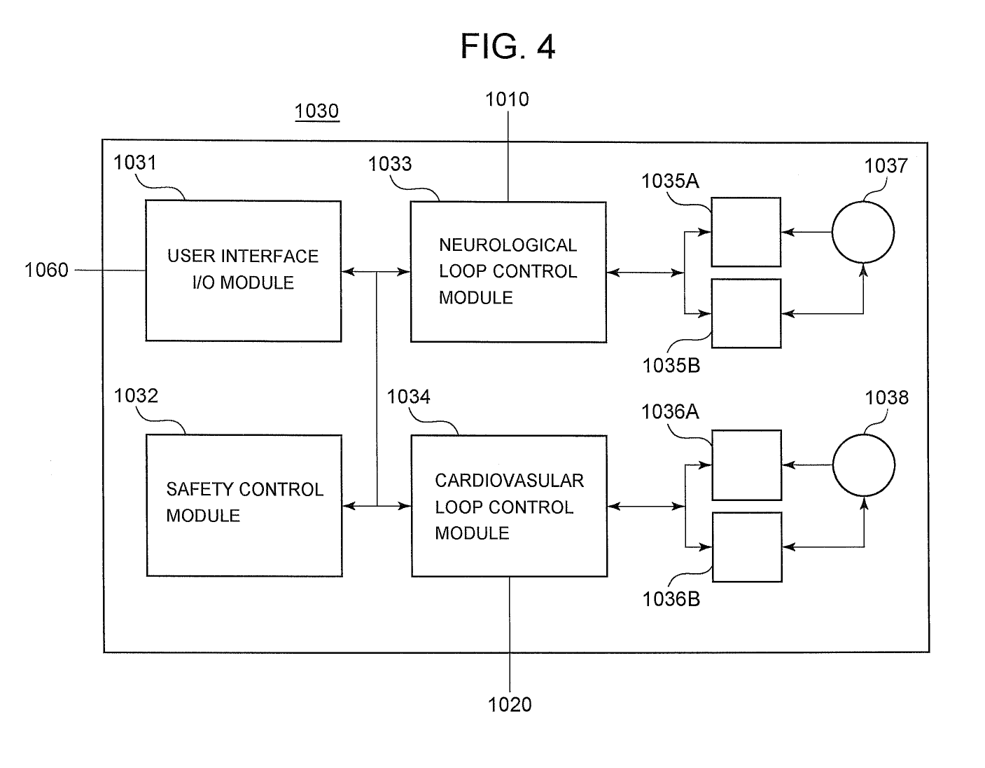

FIG. 4 is a schematic diagram illustrating a preferred embodiment of the central monitoring and delivering control unit 1030 according to the current invention, One preferred embodiment further includes a user interface input/output (I/O) module 1031, a safety control module 1032, a neurological loop control module 1033, a cardiovascular loop control module 1034, an anesthetic dose determination module 1035A, an anesthetic dose recording module 1035B, an individual anesthetic dose table 1037, a vasoactive agent dose determination module 1036A, a vasoactive dose recording module 1036B and an individual vasoactive dose table 1038. These modules of the central monitoring and delivering control unit 1030 are implemented either by software or hardware. Furthermore, these modules are organized in an exemplary manner and are not limited to the disclosed organizations. In this regard, another embodiment of the central monitoring and delivering control unit 1030 lacks certain modules such as the anesthetic dose recording module 1035B and the vasoactive dose recording module 1036B.

In another embodiment, the individual anesthetic dose table 1037 and the individual vasoactive dose table 1038 are organized in different ways. Although the drawing illustrates a single pair of the individual anesthetic dose table 1037 and the individual vasoactive dose table 1038, these two tables are optionally combined into a single table. Furthermore, the two tables are separately allocated for each patient or commonly shared with separate individual entries among the patients.

The neurological loop control module 1033 generally controls the processing flow of the information in relation to the monitored anesthetic level and the general anesthetic administration. The neurological loop control module 1033 receives information from the neurological control unit 1010. In particular, the anesthetic level control unit 1014 sends information including the anesthetic agent delivery commands and the monitored anesthetic level so that the neurological loop control module 1033 ascertains that the medically acceptable level of anesthetic continues during surgery. The neurological loop control module 1033 calls a predetermined set of modules such as the anesthetic dose determination module 1035A and the anesthetic dose safety module 1035B with the received information. Although the exemplary drawing of FIG. 4 illustrates only two modules or subroutines to be called by the neurological loop control module 1033, the number of modules is not limited to two and other modules exist in other implementations.

The anesthetic dose determination module 1035A has multiple purposes depending upon an implementation in the preferred embodiment according to the current invention. One general purpose is to provide redundancy for safety while another is to determine a precise dose for a particular individual patient. As described with respect to FIG. 2, in one preferred embodiment, the neurological data analysis unit 1012 such as BIS monitor generates a BIS value. Based upon the BIS value, the anesthetic level control unit 1014 generates the anesthetic delivery commands indicating a predetermined dose to the anesthetic agent delivery unit 1050. As to the redundancy, in one preferred embodiment, the anesthetic dose determination module 1035A independently determines the amount of a general anesthetic dose based upon the anesthetic level value. The two independent dose determination mechanisms for a general anesthetic provide redundancy in the system and improve safety when one of the dose determination mechanisms fails.

The anesthetic dose determination module 1035A improves safety for a particular patient by determining a precise anesthetic dose based upon the individual anesthetic dose table 1037. Prior to surgery, the individual anesthetic dose table 1037 is created to limit certain conditions for a particular individual patient. For example, the information includes age, genders, weight, cardiac parameters, normal vital signs and so on. The information optionally includes additional information such as a maximum amount of anesthetic dose. For inhalational agents, it includes information on agent specific MAC (Mean Alveolar Concentration) in volumes percent of anesthetic gas necessary to prevent 50% of patients from moving in response to a surgical stimulus. For intravenous infusion agents such as Propofol, it includes a weight based dosage range in mg/kg, which serve as ‘guardrails’ as in the ‘Alaris’ microprocessor controlled intravenous infusio pumps, made by Cardinal Health. Finally, the anesthetic dose determination module 1035A compares its anesthetic dose to that in the anesthetic delivery command which was generated by another component of the system and reports a difference if any to the safety control module 1032 via the neurological loop control module 1033.

The anesthetic dose recording module 1035B maintains in the individual anesthetic dose table 1037 information on a general anesthetic for a particular patient 1000. After ascertaining that the general anesthetic has been actually delivered to the patient according to the anesthetic delivery command, the anesthetic dose recording module 1035B continuously records the information on the actually delivered time, amount and type of a general anesthetic for a particular patient in the individual anesthetic dose table 1037. Each entry of the recorded information also has a patient ID to specify a particular patient. As will be later described in detail, the anesthetic dose recording module 1035B retrieves information for a particular patient as specified in an anesthetic record retrieving command from the safety control module 1032 via the neurological loop control module 1033.

The cardiovascular loop control module 1034 generally controls the processing flow of the information in relation to the cardiovascular homeostasis and the vasoactive agent administration. The cardiovascular loop control module 1034 receives information from the cardiovascular control unit 1020. In particular, the cardiac parameter control unit 1024 sends information including the vasoactive agent delivery commands and the monitored cardiac parameters so that the cardiovascular loop control module 1034 ascertains the medically acceptable level of anesthetic continues during a surgery. The cardiovascular loop control module 1034 calls a predetermined set of modules such as the vasoactive agent dose determination module 1036A and the vasoactive dose safety module 1036B with the received information. Although the exemplary drawing of FIG. 4 illustrates only two modules or subroutines to be called by the cardiovascular loop control module 1034, the number of modules is not limited to two and other modules exist in other implementations.

The vasoactive agent dose determination module 1036A has multiple purposes depending upon an implementation in the preferred embodiment according to the current invention. One general purpose is to provide redundancy for safety while another is to determine a precise dose for a particular individual patient. As described with respect to FIG. 3A, in one preferred embodiment, the cardiac data analysis unit 1022 generates a set of cardiac parameter values, and the cardiac parameter control unit 1024 generates the vasoactive agent delivery commands indicating a predetermined dose to the vasoactive agent delivery unit 1040 based upon the cardiovascular homeostatic level in terms of the cardiac parameters that are determined by the cardiac data analysis unit 1022. As to the redundancy, in one preferred embodiment, the vasoactive agent dose determination module 1036A independently determines the amount of a vasoactive agent dose based upon the corresponding cardiac parameter value. The two independent determination mechanisms for a vasoactive agent dose provide redundancy in the system and improve safety when one of the dose determination mechanisms fails.

The vasoactive agent dose determination module 1036A improves safety for a particular patient by determining a precise vasoactive drug dose based upon the individual vasoactive dose table 1038. Prior to surgery, the individual vasoactive dose table 1038 is created to limit certain conditions for a particular individual patient. For example, the information includes information such as a maximum dose of a particular vasoactive agent. For intravenous infusion agents such as Dobutamine, Phenylpehrine, Nitroglycerine, Nicardipine, Esmolol, and similar drugs, it includes a weight based dosage range in mg/kg, which serve as ‘guardrails’ as in the ‘Alaris’ microprocessor controlled intravenous infusion pumps, made by Cardinal Health. For an inhalational vasoactive agent such as nitric oxide (NO), the dosage ranges from 1-100 parts per million. Finally, the vasoactive agent dose determination module 1036A compares its vasoactive agent dose to that in the vasoactive agent delivery command which was generated by another component of the system and reports any difference to the safety control module 1032 via the cardiovascular loop control module 1034.

The vasoactive dose recording module 1036B maintains in the individual vasoactive dose table 1038 information on each dose of predetermined vasoactive drugs for a particular patient 1000. After ascertaining that the vasoactive agent has been actually delivered to the patient according to the vasoactive agent delivery command, the vasoactive dose recording module 1036B continuously records the information in the individual vasoactive dose table 1038. Each entry of the recorded information includes at least an amount, time and type of each vasoactive agent delivered to a particular patient as well as a patient ID. As will be later described in detail, the vasoactive dose recording module 1036B retrieves information for a particular patient as specified in a vasoactive record retrieving command from the safety control module 1032 via the cardiovascular loop control module 1034.

The user interface input/output (I/O) module 1031 handles the interface with the user interface unit 1060, which has an input and or output capabilities to send or receive signals to and from an operator. For example, after either the anesthetic dose safety module 1035B or the vasoactive dose safety module 1036B determines that a warning must be given to an operator in response to a predetermined dangerous situation, the user interface I/O module 1031 receives an operator-warning signal via the neurological loop control module 1033 or the cardiovascular loop control module 1034. The user interface I/O module 1031 sends the operator-warning signal to the user interface unit 1060 so that the user interface unit 1060 generates an audio and or visual warning signal. In one implementation, the user interface I/O module 1031 sends the operator-warning signal as a high-priority signal or via interrupt to the user interface unit 1060 so that the audio visual signal is immediately generated even if other tasks are pending. In response to the operator-warning signal, the operator acknowledges the warning signal or responds to the warning signal through the input device such as a touch screen or a keyboard of the user interface unit 1060.

The user interface I/O module 1031 receives the input from the user interface unit 1060 and determines the priority of the input signal. If it is determined as a high-priority signal, the user interface I/O module 1031 interrupts the neurological loop control module 1033 and or the cardiovascular loop control module 1034 for immediate processing. Otherwise, the user interface I/O module 1031 places the user input signal on an appropriate cue of tasks to be later processed by the neurological loop control module 1033 and or the cardiovascular loop control module 1034. The priority signal processing is not limited to interrupts and or cues and further includes other commonly known software or hardware implementations.

In addition, the user interface input/output (J/O) module 1031 sends a predetermined set of information for display and updates it. In addition to the anesthetic level and the cardiac parameters, the user optionally selects to display the predetermined information among the vital sign and certain cardiac parameters. Furthermore, the user also optionally selects a manner in which the selected information is displayed. For example, the selected information is displayed in its values in digits or in a graphical representation such as a three-dimensional vector. Lastly, the user optionally selects the update frequency such as a predetermined time interval or in a real-time.

The safety control module 1032 ascertains safety of the patient 1000 under a general anesthetic by performing a predetermined set of comprehensive analyses. It is well known that all of the anesthetic or sedative-hypnotic agents substantially affect the patient's cardiovascular state by virtue of their depressant actions on the autonomic nervous system, myocardial contractility, systemic vascular resistance, preload and cardiac output. Furthermore, manipulations by surgeons also cause the myriad other physiological events during operation. In order to access the above described risks, the safety control module 1032 performs the analyses using the information in the individual anesthetic dose table 1037 and the individual vasoactive dose table 1038. To obtain the information, the safety control module 1032 sends a request to either the neurological loop control module 1033 or the cardiovascular loop control module.

As described before, the individual anesthetic dose table 1037 and the individual vasoactive dose table 1038 store a predetermined set of information on each of the patients 1000. The individual anesthetic dose table 1037 contains information on age, genders, weight, cardiac parameters, normal vital signs, the monitor anesthetic level and the associated time, and the amount and time of the general anesthetic delivery for each patient. The individual vasoactive dose table 1038 contains information on the monitor cardiac parameters and the associated time and the amount and time of the vasoactive agent delivery for each patient.

The safety control module 1032 generates an information request to obtain information for a particular individual patient from the individual anesthetic dose table 1037 and the individual vasoactive dose table 1038. The information request is generated based upon a certain predetermined rule such as a time interval and or a certain signal such as an operator-warning signal from the anesthetic dose safety module 1035B or the vasoactive dose safety module 1036B. Alternatively, as described with respect to FIG. 2, the anesthetic level control unit 1014 compares the BIS value to a predetermined set of threshold values such as the lowest BIS threshold value of 40 and the highest BIS threshold value of 60, If one of certain situations is identified that the anesthetic level is too deep or shallow, the anesthetic level control unit 1014 transmits the BIS value to the central monitoring and delivering control unit 1030 for performing a further clinical analysis. Similarly, as also described with respect to FIG. 3A, the cardiac parameter control unit 1024 compares the cardiac parameter values to a predetermined set of threshold vector component values. The respective threshold value in {EI, DI, MAP, E-M, Heartrate} or its transformed value in the invasive vector space H′, is set to +20% or −20% of what is normal for a particular patient. For example, if one of certain critical situations is identified that the Preload value is too high or low, the cardiac parameter control unit 1024 transmits the Preload value to the central monitoring and delivering control unit 1030 for performing a further clinical analysis. As specified in its information request for obtaining certain information, the safety control module 1032 receives the relevant information from the individual anesthetic dose table 1037 and or the individual vasoactive dose table 1038 in response to its request.

Based upon the received information, the safety control module 1032 performs a relevant analysis in response to the above described risk factors. For example, the safety control module 1032 independently determines an anesthetic agent dose and a vasoactive agent dose based upon the current monitored conditions and any other limitations of the patient in the received information. Preferably, the independent determination is based upon an algorithm that takes additional information into account and or that is more conservative in determining the dose than the algorithm used in the anesthetic level control unit 1014 or the cardiac parameter control unit 1024. To ascertain the safety, the safety control module 1032 compares the above independently determined doses to the actually delivered doses for a particular individual patient. The safety control module 1032 takes certain actions based upon the difference in value according to the predetermined rules. For example, if the difference is within a predetermined threshold value, the safety control module 1032 sends an operator-warning signal indicating a minor discrepancy to the user interface unit 1060 via the user interface I/O module 1031 On the other hand, if the difference is over the predetermined threshold value, the safety control module 1032 also sends a stop delivery signal to the vasoactive agent delivery unit 1040 and or the anesthetic agent delivery unit 1050 so that the delivery of the anesthetic agent and or the vasoactive agent is immediately terminated. The safety control module 1032 also sends an operator-warning signal indicating the major discrepancy and or disengagement of the delivery unit(s) 1040, 1050 to the user interface unit 1060 via the user interface I/O module 1031. In the latter situation, the automatic pilot mode is effectively disengaged in the delivery units 1040 and 1050.

The above independent dose analysis provides additional redundancy in safety. As illustrated in FIGS. 2 and 3, the anesthetic level control unit 1014 and the cardiac parameter control unit 1024 respectively determine the anesthetic agent delivery amount and the vasoactive agent delivery amount. Subsequently, the safety control module 1032 independently determines an anesthetic agent dose and a vasoactive agent dose and compares them for safety. This double-checking mechanism is further strengthened by additional monitoring mechanisms such as monitoring the actually delivered amount of the anesthetic and vasoactive agents at the patient 1000. The safety control module 1032 also optionally oversees the dosage history over a long period of time to further increase the safety level.

The above described functions of the modules in FIG. 4 are merely exemplary and are not limited to the disclosed functions. In addition, the organizations of the modules are also not limited to the disclosed ones. In other words, the disclosed functions are optionally organized in manners that are different from the disclosed configuration. For example, different organizations include functional redundancy among the modules.

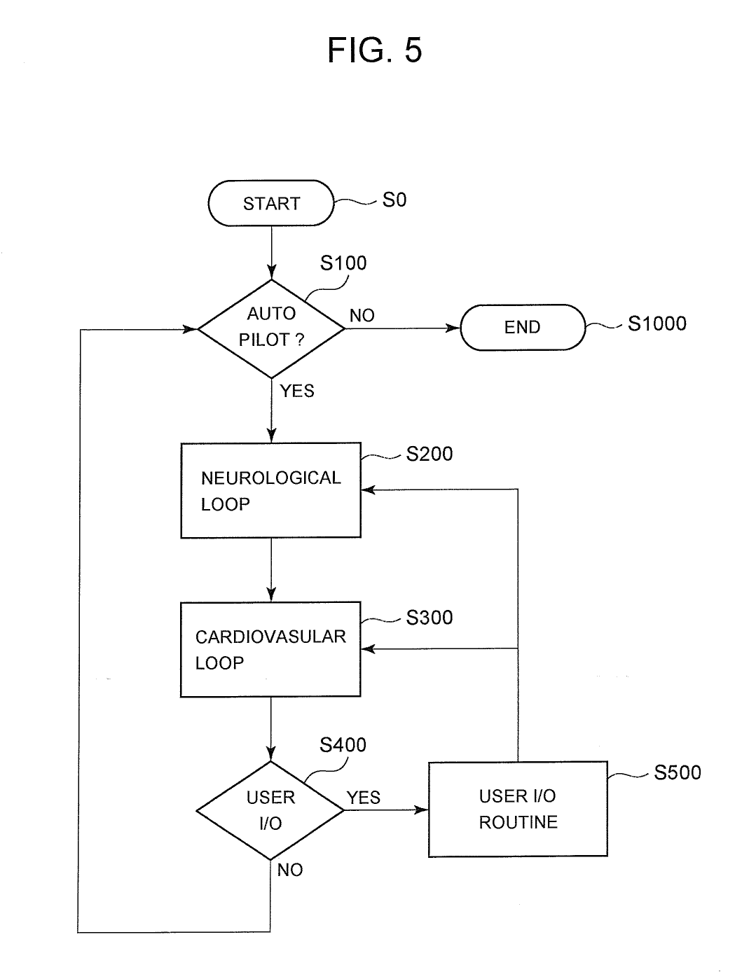

Now referring to FIG. 5, a flow chart illustrates steps involved in a preferred process of administering a general anesthetic agent based upon neurological parameters while maintaining cardiovascular homeostasis based upon cardiac parameters with minimal human intervention according to the current invention. In general, the preferred process generally includes the steps associated with administering a general anesthetic to maintain the patient in a neurologically depressed state while administering vasoactive agents to maintain his or her cardiovascular homeostasis according to cardiac parameters preferably with the least amount of human intervention during surgery. The overarching goal is to assure that the level of neurological depression is minimally necessary, yet sufficient, and optimal. Similarly, the permissible level of vasoactive agent infusion support is minimally necessary, yet sufficient and optimal to maintain vital organ perfusion homeostasis at the above optimal level of neurological depression. Thus, the preferred process responds to changes in anesthesia level and vasoactive infusion support to achieve homeostasis by actually titrating to defined physiological endpoints in a way that is more rapid and accurate than a skilled care provider can achieve. The neuro-cardiovascular control process of the current invention is analogous to an automatic pilot control in aviation or a cruise control in automobile after the patient is properly induced by an anesthesiologist. An operator or anesthesiologist relies upon the neuro-cardiovascular cruise control until either the system disengages itself upon identifying a critical condition or the operator triggers a termination signal at any time as if stepping on the break to terminate the cruise control in an automobile.

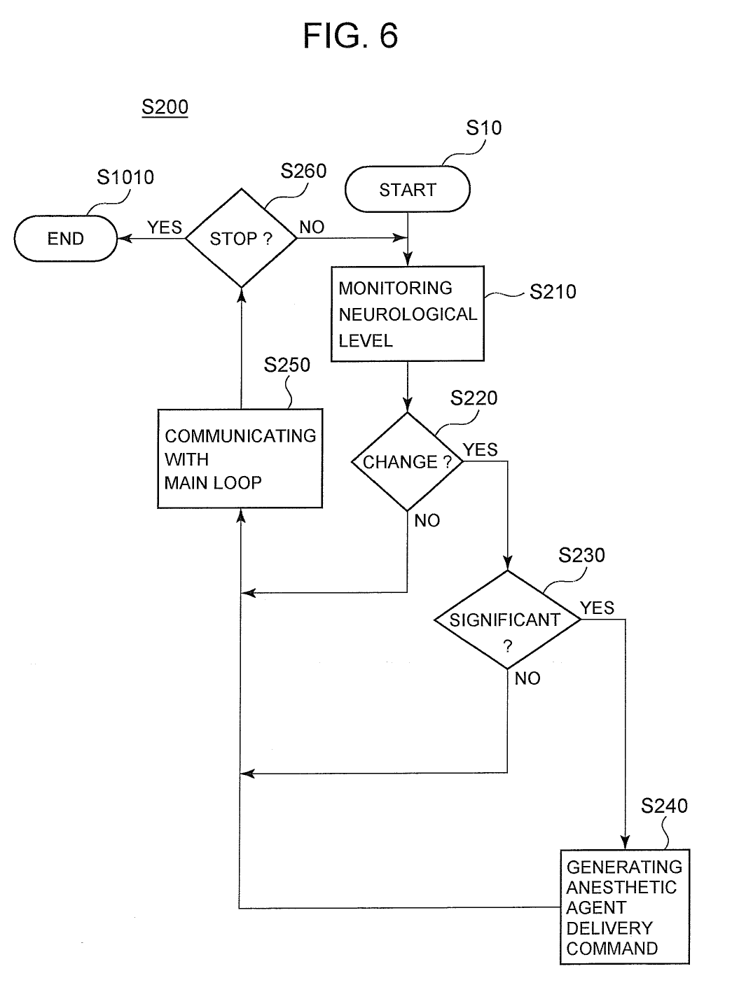

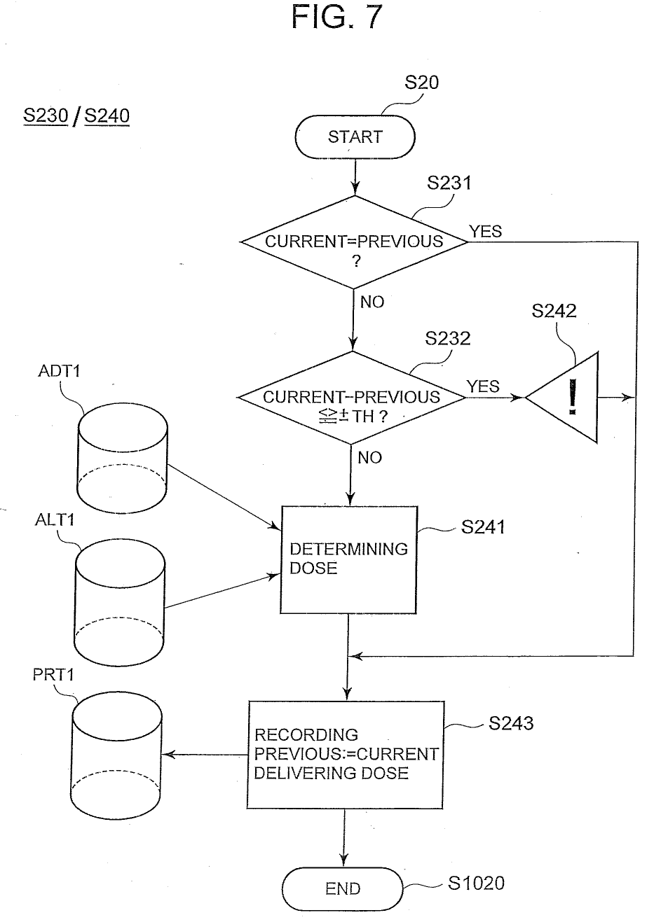

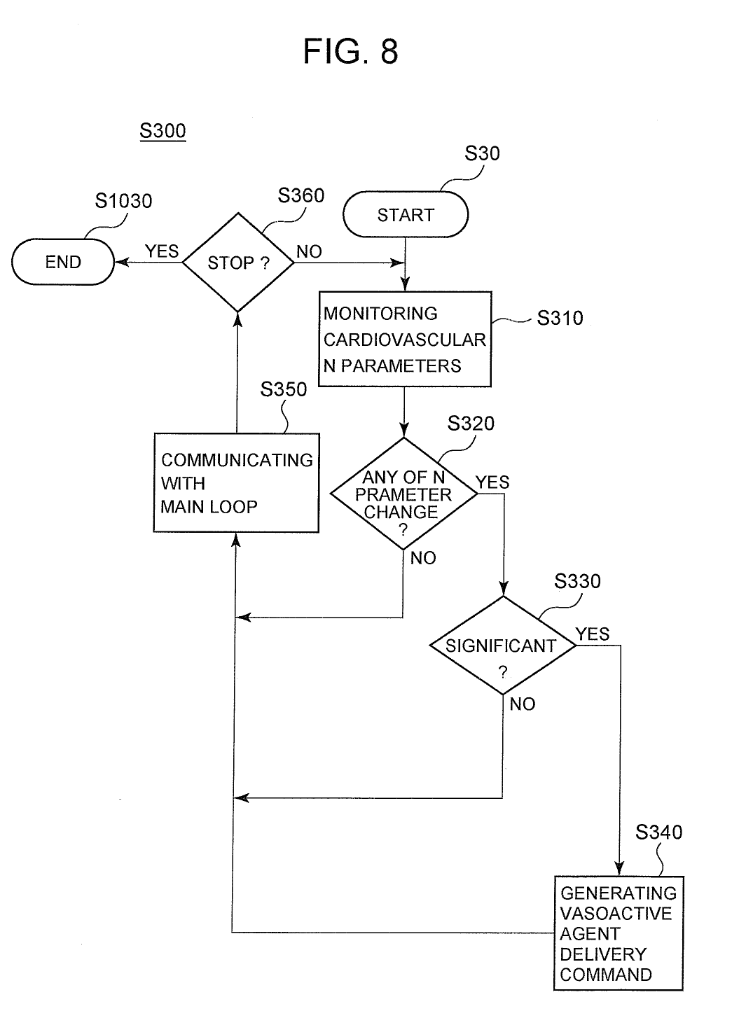

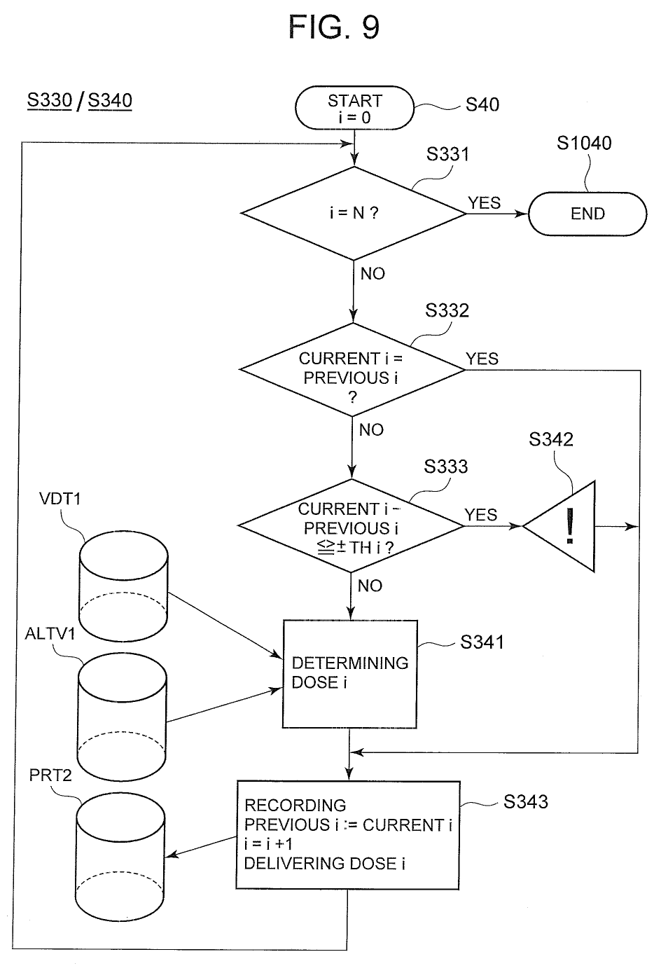

In particular, the neuro-cardiovascular control process of the current invention has loops including a main loop and two additional loops that are nested in the main loop. Although the details of the double-nested loops are not illustrated in this flow chart, each of the two loops resides in a neurological loop S200 and a cardiovascular loop S300, and these two nested loops are within the neuro-cardiovascular control main loop, which starts in a step S0 and repeats itself until it is determined in a step S100 to stop in a step S1000 as illustrated in FIG. 5. After the patient is properly induced by an anesthesiologist in a step S0, the neuro-cardiovascular control process determines if an auto-pilot mode has been activated in a step S100. The auto-pilot mode according to the current invention means a mode of operation in which the neurological anesthetic control process and the cardiovascular control process are each performed with minimal human intervention until a certain predetermined condition or event occurs. If it is determined in the step S100 that the auto-pilot mode has not been activated, the neuro-cardiovascular control process is terminated in an end step S1000. The termination is determined based upon a variety of mechanisms that involves software and or hardware implementations. On the other hand, if it is determined in the step S100 that the auto-pilot mode has been activated, the neuro-cardiovascular control process proceeds to a neurological loop S200, where a first nested loop is followed as will be described with respect to FIGS. 6 and 7. Similarly, the neuro-cardiovascular control process also proceeds to a cardiovascular loop S300, where a second nested loop is followed as will be described with respect to FIGS. 8 and 9. Although the two nested loops S200 and S300 are illustrated as two consecutive steps in the flow chart of FIG. 5, these two steps are optionally activated in a simultaneous manner. The implementations for the simultaneous loops include software and or hardware technologies. For example, two processes are respectively spawned for the two nested loops S200 and S300 from the main loop, and they communicated among themselves via interprocess communication technique at run time.

Still referring to FIG. 5, the main loop of the neuro-cardiovascular control process additionally includes a step S400 to determine whether or not an input or output has been generated to and from a user or an operator. If it is determined in the step S400 that a user has input any information via the user interface unit 1060, the neuro-cardiovascular control process proceeds to a User Input Output (I/O) routine in a step S500 for processing the user input. For example, the User Input I/O routine in the step S500 processes a user request to disengage the automatic pilot mode by triggering a termination signal to the neurological loop in the step S200 and or the cardiovascular loop in the step S300. The User Input I/O routine also initiates a predetermined mechanism such as a flag to be in a non-automatic pilot mode for the step S100. Another example is that either of the neurological loop in the step S200 or the cardiovascular loop in the step S300 returns an output message such as a warning message or an updated information on the patient's condition such as an updated Afterload value to be displayed. The User Input I/O routine in the step S500 processes the output to be displayed by the user interface unit 1060 such as a monitor. On the other hand, if it is determined in the step S400 that a user has not input any information, the neuro-cardiovascular control process loops back to the step S100 to repeat the process.

As described above, the main loop nests the two inner loops in the steps S200 and S300 to control the general high-level flow of the cardiovascular control process. The high-level flow control is preferably implemented to have the main loop and the two nested loops S200 and S300 are independent of each other. In other words, the three loops are each repeated independent of the each other until they are separately terminated. However, as will be further described later in detail, these three loops communicate with each other to pass certain information to possibly affect the operation of predetermined steps in another loop or even to terminate another loop.

Now referring to FIG. 6, a flow chart illustrates steps involved in the process of administering a general anesthetic agent based upon neurological parameters with minimal human intervention according to the current invention. In general, the process generally includes the steps associated with the neurological loop in the step S200 for administering a general anesthetic to maintain the patient in a neurologically depressed state according to monitored neurological parameters preferably with the least amount of human intervention during surgery. As described above with respect to FIG. 5, the step S200 contains the nested loop, which starts in a step S110 and repeats the neurological loop S200 until it is determined in a step S260 to stop in a step S1010 as illustrated in FIG. 6.

The neurological nested loop S200 further includes the steps associated with monitoring a neurological parameter and administering a general anesthetic based upon the monitored neurological parameter to maintain a desirable level of neurological depression with minimal human intervention according to the current invention. After the neurological nested loop S200 is initiated in a start step S10 when it is called from the main loop of the neuro-cardiovascular control process, a predetermined set of neurological parameter data is collected in a step S210 by monitoring a neurological level of a patient. The monitoring technique is generally non-invasive in a preferred process. One example of a commercially available technology for non-invasively monitoring a neurologically depressed state of a patient is a bispectral index (BIS) monitor from Aspect Medical Systems. The BIS monitor continually analyses EEG from a sensor placed on the patient's forehead during general anesthesia to assess the level of consciousness in a single digit ranging from 0 to 100 without any unit. The manufacturer recommends a BIS value between 40 and 60 in for an ordinary level of general anesthesia. In preferred embodiment, the BIS value is maintained near 55 or between 50 and 60. Although a BIS value is used in a preferred embodiment, the neuro-cardiovascular control process of the current invention is not limited to this implementation and other values are used to monitor the neurologically depressed level in a patient.

Still referring to FIG. 6, the above described monitored value is analyzed in steps S220 and S230 to determine if the neurologically depressed level should be altered in the patient. Initially, it is determined if the currently monitored value is different from the previously monitored value in the step S220. If there is no difference in the monitored values, the neuro-cardiovascular control process proceeds back to the step S210 to repeat the monitoring until there is a change or it is stopped in a step S260. On the other hand, if it is determined that there is a difference between the monitored values in the step S220, it is further determined whether or not the difference is significant by performing a predetermined routine in a step S230. One such routine compares the difference to a predetermined threshold value. For example, the threshold value is 50 or so for the use with a BIS value. Another routine determines the significance in a cumulative manner over a time. If the difference is determined to be insignificant in the step S230, the neuro-cardiovascular control process proceeds back to the step S210 to repeat the monitoring until there is a significant change or it is stopped in the step S260.

If the step S230 confirms a significant difference, the neuro-cardiovascular control process proceeds to a step S240, where an anesthetic agent delivery command is generated. The delivery commands include data to specify an agent to be delivered, a pump number, a reservoir number, a rate of delivery and a total amount of the agent. In addition, a warning message is also generated in certain situations as will be described with respect to FIG. 7. As described with respect to FIG. 2, in one preferred process, the central monitoring and delivering control unit 1030 generates the anesthetic delivery commands to the anesthetic agent delivery unit 1050 based upon the anesthetic level that is monitored through the neurological control unit 1010 in one preferred process. Alternatively, the neurological control unit 1010 generates the anesthetic delivery commands to the anesthetic agent delivery unit 1050 based upon the anesthetic level that is monitored through the neurological control unit 1010 in an alternative process. In another alternative process, the two units 1010 and 1030 each generate the anesthetic delivery commands using the monitored data according to a different algorithm and or additional data.

Regardless of the above described paths, the neuro-cardiovascular control process performs a communication routine in a step S250 and a stop determination routine in a step S260. In the communication routine in a step S250, certain information is returned to the main loop in the neuro-cardiovascular control process. For example, if an anesthetic agent delivery command is generated, the associated information in the command is passed to the main loop via a predetermined mechanism including global variables, inter-process communication or interrupts. This allows the main loop to further distribute the information to a different loop or module. Lastly, the stop determination routine in a step S260 determines whether or not the neurological nested loop S200 is terminated based upon a result of the analyses within the neurological nested loop S200 or information that is passed from the outside to the neurological nested loop S200. The analysis result is communicated via a predetermined mechanism including global variables, inter-process communication or interrupts.

Now referring to FIG. 7, a flow chart illustrates further steps involved in the detailed control process of determining a general anesthetic agent dose based upon neurological parameters with minimal human intervention according to the current invention. In general, the process generally includes the detailed steps associated with the neurological level change detection in the step S230 and the anesthetic agent delivery command generation in the step S240 for processing the monitored neurological parameters. The detailed steps as illustrated in FIG. 7 are not necessarily limited to the steps S230 and S240 and are optionally included in other steps or called from other steps.

Still referring to FIG. 7, the further detailed process is a routine and not a loop. A step S231 compares the current and previously monitored neurological values after the detailed steps are invoked in a step S20. If it determines no difference in the current and previously monitored neurological values, the detailed control process proceeds to a step S243. On the other hand, if it detects a difference between the current and previously monitored neurological values, the difference is further compared against a predetermined threshold value in a step S232. The threshold value depends upon a number of factors including a monitoring frequency, a monitoring value type and a risk tolerance level. When there is a significant or critical change beyond the threshold value over one predetermined monitoring period between the current and previously monitored neurological values, a warning message is generated in a step S242 depending upon a level of significance. The warning message is displayed to the operator and or used by the main loop of the neuro-cardiovascular control process. On the other hand, if the step S232 determines that the change is within a predetermined threshold value over one predetermined monitoring period between the current and previously monitored neurological values, an anesthetic agent dose is determined for a delivery command in a step S241.

To minimize the associated risks, the dose determining step S241 uses the monitored information as well as other external data for accurately determine a proper dosage. For example, a dose is determined according to a predetermined dosage table ADT1 of a particular anesthetic agent based upon physical characteristics of a patient. Furthermore, as described above, the anesthetic agent dose is determined by a central unit or a local unit using a different algorithm as specified in an algorithm table ALT1. For example, a dose selected from the dosage table is optionally modified by an algorithm. In case of any conflict or a difference in the determined anesthetic agent dose, the anesthetic agent dose determining routine in the step S241 resolves the issue by selecting one of the two dosages and or issuing a warning message without selecting one dosage. In any case, the determined dose and or the warning message are returned to a calling routine via a predetermined mechanism such as global variables, inter-process communication or interrupts. Lastly, the currently monitored neurological parameter data and the determined dose are recorded in a patient table PRT1 in a step S243. Additionally, the currently monitored data now becomes the previously monitored data. The above external data are merely illustrative, and the current invention optionally utilizes other data common in the relevant field of anesthesiology. In addition, these data tables are also accessible by other modules, loops or routines as it is necessary or desirable.

Now referring to FIG. 8, a flow chart illustrates steps involved in the process of administering a vasoactive agent based upon cardiovascular parameters with minimal human intervention according to the current invention. In general, the process generally includes the steps associated with the cardiovascular loop in the step S300 for administering a vasoactive agent to maintain the patient in cardiovascularly stable state according to monitored cardiovascular parameters preferably with the least amount of human intervention during surgery. As described above with respect to FIG. 5, the step S300 contains the nested loop, which starts in a step S30 and repeats the cardiovascular loop S300 until it is determined in a step S360 to stop in a step S1030 as illustrated in FIG. 8.

The cardiovascular nested loop S300 further includes the steps associated with monitoring cardiovascular parameters and administering a vasoactive agent based upon the monitored cardiovascular parameter to maintain a desirable cardiovascular homeostasis with minimal human intervention according to the current invention. After the cardiovascular nested loop S300 is initiated in a start step S30 when it is called from the main loop of the neuro-cardiovascular control process, a predetermined set of cardiovascular data is collected in a step S310 by monitoring cardiovascular parameters of a patient. The monitoring technique is generally non-invasive in a preferred process. Although there is not a commercially available technology for non-invasively monitoring cardiovascular parameters of a patient, U.S. Pat. No. 7,054,679 issued to Hirsh discloses the non-invasively measuring the hemodynamic state of a patient based upon cardiac cycle period, electrical-mechanical interval, mean arterial pressure, ejection interval, and diastolic filling interval. The above measured cardiovascular values are converted to a predetermined set of cardiac parameters. The details of the non-invasive hemodynamic measurement and conversion techniques will be further discussed in detail later in this specification.

Still referring to FIG. 8, the above described converted values are analyzed in steps S320 and S330 to determine if a particular vasoactive agent should be administered to the patient. Initially, it is determined if the currently monitored cardiac value is different from the corresponding previously monitored cardiac value in the step S320. If there is no difference in the monitored values, the cardiovascular control process proceeds back to the step S310 to repeat the monitoring until there is a change or it is stopped in a step S360. On the other hand, if it is determined that there is a difference between the monitored values in the step S320, it is further determined whether or not the difference is significant by performing a predetermined routine in a step S330. One such routine compares the difference to a predetermined threshold value. For example, the threshold value for Preload is approximately ±20% of the normal Preload value for a particular patient. Another routine determines the significance in a cumulative manner over a time. If the difference is determined to be insignificant in the step S330, the neuro-cardiovascular control process proceeds back to the step S310 to repeat the monitoring until there is a significant change or it is stopped in the step S360. In the above, although only one cardiovascular parameter is described, a series of predetermined cardiovascular parameters is analyzed using a corresponding data. The serial analyses are optionally implemented as a loop for each of these cardiovascular parameters.

If the step S330 confirms a significant difference, the neuro-cardiovascular control process proceeds to a step S340, where a vasoactive agent delivery command is generated. The delivery commands include data to specify an agent to be delivered, a pump number, a reservoir number, a rate of delivery and a total amount of the agent. In addition, a warning message is also generated in certain situations as will be described with respect to FIG. 9. As described with respect to FIG. 3A, in one preferred process, the central monitoring and delivering control unit 1030 generates the vasoactive agent delivery commands to the vasoactive agent delivery unit 1040 based upon the cardiac parameter that is monitored through the vasoactive control unit 1020 in one preferred process. Alternatively, the vasoactive control unit 1020 generates the vasoactive agent delivery commands to the vasoactive agent delivery unit 1040 based upon the cardiac parameters that are monitored through the vasoactive control unit 1020 in an alternative process. In another alternative process, the two units 1020 and 1030 each generate the vasoactive delivery commands using the monitored data according to a different algorithm and or additional data. In the above, although only one vasoactive agent delivery command is described, a series of predetermined vasoactive agent delivery commands is generated using a corresponding data under certain circumstances. The serial generations are optionally implemented as a loop for generation of each of the vasoactive agent delivery commands.

Regardless of the above described paths, the neuro-cardiovascular control process performs a communication routine in a step S350 and a stop determination routine in a step S360. In the communication routine in a step S350, certain information is returned to the main loop in the neuro-cardiovascular control process. For example, if a vasoactive agent delivery command is generated, the associated information in the command is passed to the main loop via a predetermined mechanism including global variables, inter-process communication or interrupts. This allows the main loop to further distribute the information to a different loop or module. Lastly, the stop determination routine in a step S360 determines whether or not the cardiovascular nested loop S300 is terminated based upon a result of the analyses within the cardiovascular nested loop S300 or information that is passed from the outside to the cardiovascular nested loop S300. The analysis result is communicated via a predetermined mechanism including global variables, inter-process communication or interrupts.

Now referring to FIG. 9, a flow chart illustrates further steps involved in the detailed control process of determining a vasoactive agent dose based upon cardiovascular parameters with minimal human intervention according to the current invention. In general, the process generally includes the detailed steps associated with the cardiac level change detection in the step S330 and the vasoactive agent delivery command generation in the step S340 for processing the monitored cardiovascular parameters. The detailed steps as illustrated in FIG. 9 are not necessarily limited to the steps S330 and S340 and are optionally included in other steps or called from other steps.

Still referring to FIG. 9, the further detailed process is a routine. After the detailed steps are invoked in a step S40, it is determined whether or not all of the predetermined N cardiac parameters are already analyzed in a step S331. If all N cardiac parameters have been already analyzed, the detailed process ends in a step S1040. Otherwise, a step S332 compares the current and previously monitored cardiac parameter values. If it determines no difference in values between the current and previously monitored cardiac parameter values, the detailed control process proceeds to a step S343. On the other hand, if it detects a difference between the current and previously monitored cardiovascular parameter values, the difference is further compared against a predetermined threshold value in a step S333. The threshold value depends upon a number of factors including a monitoring frequency, a monitoring value type and a risk tolerance level. When there is a significant or critical change beyond the threshold value over one predetermined monitoring period between the current and previously monitored cardiovascular parameter values, a warning message is generated in a step S342 depending upon a level of significance. The warning message is displayed to the operator and/or used by the main loop of the neuro-cardiovascular control process. On the other hand, if the step S333 determines that the change is within a predetermined threshold value over one predetermined monitoring period between the current and previously monitored neurological values, a vasoactive agent dose is determined for a delivery command in a step S341 In the above, although the dose determination is described only for one vasoactive agent, a series of predetermined vasoactive agent doses is generated using a corresponding data under certain circumstances. The serial determinations are optionally implemented as a loop for generation of each of the vasoactive agent doses.

To minimize the associated risks, the dose determining step S341 uses the monitored information as well as other external data for accurately determining a proper dosage. For example, a dose is determined according to a predetermined dosage table ADT1 of a particular vasoactive agent i based upon physical characteristics of a patient. For example, a dose selected from the dosage table is optionally modified by an algorithm. Furthermore, as described above, the vasoactive agent dose is determined by a central unit or a local unit using a different algorithm as specified in an algorithm table ALTV1. In case of any conflict or a difference in the determined vasoactive agent dose, the vasoactive agent dose determining routine in the step S341 resolves the issue by selecting one of the two dosages and or issuing a warning message without selecting one dosage. In any case, the determined dose and or the warning message are returned to a calling routine via a predetermined mechanism such as global variables, inter-process communication or interrupts.

Lastly, the currently monitored cardiovascular parameter data and the determined vasoactive dose are recorded in a patient table PRT2 in a step S343. Additionally, the currently monitored data now becomes the previously monitored data. The above external data are merely illustrative, and the current invention optionally utilizes other data common in the relevant field of anesthesiology. In addition, these data tables are also accessible by other modules, loops or routines as it is necessary or desirable. The index i is incremented by one to keep track of a number of the cardiac parameters that have been already processed in the step S343.

The above described detailed steps are repeated until it is determined that all of the predetermined N cardiac parameters are already analyzed in a step S331. If all N cardiac parameters have been already analyzed, the detailed process ends in the step S1040.

Now referring to FIG. 10, the user interface unit 1060 of the present invention further includes a monitoring device 52, which displays the output cardiac parameters as a single three dimensional vector 64 in a three dimensional space 56 as defined by the three dimensional axes 58, 60 and 62 on a screen 54. The three dimensions 58, 60 and 62 respectively represent Preload, Afterload and Contractility or their equivalents based upon either invasive or non-invasive measurements. The projections 66, 68 and 70 of the vector 64 on the axes 58, 60 and 62 respectively represent a value for the patient's Preload, Afterload and Contractility. The three dimensional graph on the screen allows a clinician to process a great deal of hemodynamic information at one glance. The display substantially improves vigilance in cardiovascular monitoring in the perioperative period.

Still referring to FIG. 10, the touch screen 54 includes other display areas and user input areas. Other display areas include a patient ID area 40 to display a predetermined set of relevant information such as a patient ID, age, gender, height (H) and weight (W). Additional patient information is optionally displayed as the operator touches the patient ID area 40. Similarly, a vital sign area 41 displays a predetermined set of relevant information such as heart rate and blood pressure (BP). Additional vital information is optionally displayed as the operator touches the vital sign area 41. As an operator touches a start button area 42 on the screen 54, the automatic pilot mode is activated to maintain a desirable anesthetic level and cardiovascular homeostasis as described above. As an operator touches a stop button area 44 on the screen 54, the automatic pilot mode is immediately disengaged for the general anesthetic administration and or the vasoactive agent administration. Furthermore, after the stop button area 44 is pressed, the operator touches the start button area 42 to resume the automatic pilot mode if the conditions for causing the warning signal have been resolved.

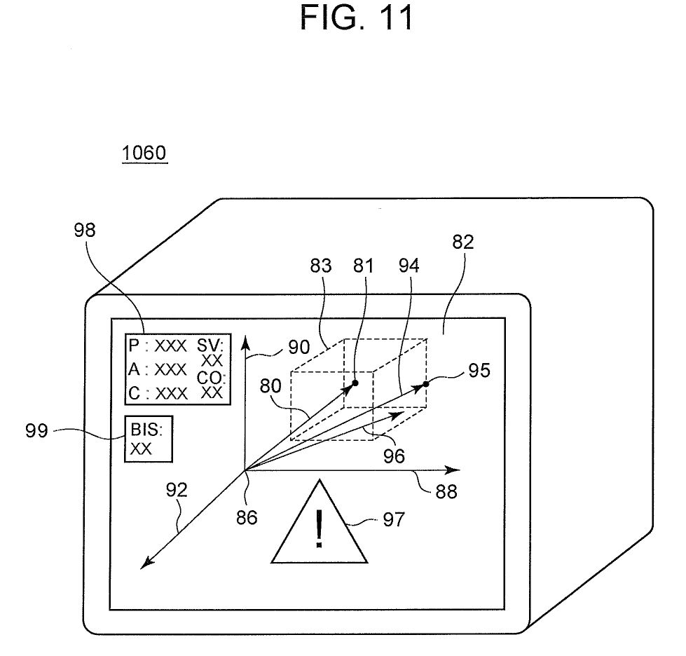

Now referring to FIG. 11, a diagram illustrates one preferred embodiment of the display in the according to the current invention. The user interface unit 1060 displays a vector 80 on the screen 82 that represents a ‘safe’ or ‘normal’ hemodynamic state or space. For instance, after the patient is sedated but before the surgery begins, the safe hemodynamic state is determined. By seeing how the vector 94 moves in real time relative to the norm vector 80, the operator or the clinician easily and visually perceives subtle changes in the patient's hemodynamic profile. The vector 94 is represented in computer graphics as a ray emanating from the origin 86. The projection of the vector 94 onto the Preload axis 88, the Afterload axis 92 and the Contractility axis 90 are optionally made distinct in different colors. Likewise, the three components of the norm vector 80 are also optionally marked to create a basis of visual comparison. A parallel vector 96 in a contrasting color is overlaid upon the hemodynamic state vector 94. The length of the parallel vector 96 represents the size of the cardiac output which is the product of the stroke volume and the heart rate. In one preferred embodiment, the user interface unit 1060 optionally displays PAC vector component values of the above described vector 94 in a first digital display area 98 and a BIS value indicative of the patient's neurologically depressed state in a second digital display area 99. In the first digital display area 98, the user interface unit 1060 also displays a stroke volume (SV) value and a cardiac output (CO) value and updates them on each respiratory cycle.

Still referring to FIG. 11, a box or a safety zone 83 is optionally drawn on the screen 82 with the center of the box at the end point 81 of vector 80. Each edge of the box 83 is either parallel or perpendicular to the axes 88, 90 and 92. The length of the edges that are parallel to the Preload axis 88 represents the safe range of the patient's Preload. By the same token, the length of the edges that are parallel to the Afterload axis 92 and the Contractility axis 90 respectively represents the safe range of the patient's Afterload and Contractility. Therefore, as long as the end point 95 of the vector 94 is within the safe zone box 83, the vital cardiac parameters are considered to be within a predetermined acceptable range.

The user interface unit 1060 provides warning signals to the operator when the vital cardiac parameters are outside of the predetermined acceptable range. For example, if either the stroke volume (SV) value or the cardiac output (CO) value exceeds 10% from a corresponding predetermined baseline value of a particular patient, a warning is provided. Similarly, if the end point 95 exits the box 83, the user interface unit 1060 provides a visual signal such as a warning icon 97 and or an audio signal such as beeps to urge an operator to take an appropriate action such as infusion of a suitable vasoactive agent for restoring cardiovascular homeostasis as indicated by the end point 95 within the box 83. The warning sign 97 is optionally blinking and includes textual messages. Furthermore, the waning sign 97 optionally prompts a stop button 44 as illustrated in FIG. 10.

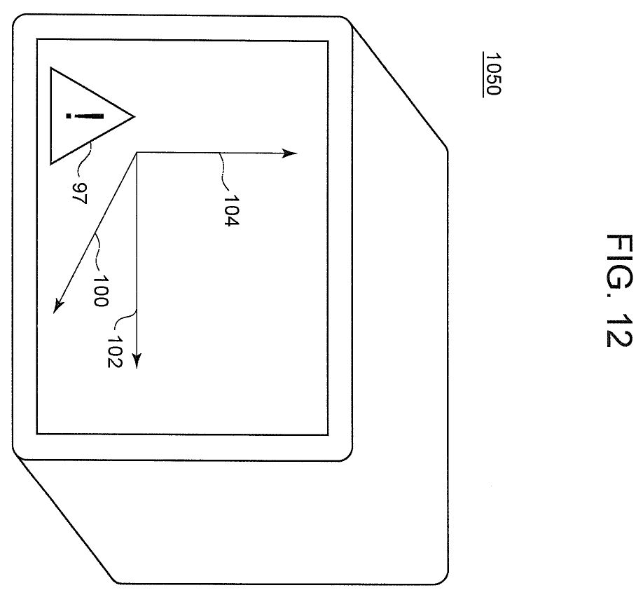

Now referring to FIG. 12, the user interface unit 1060 displays the deviation of the hemodynamic state vector from a physiological norm as indicative of an amount of physiological stress in one preferred embodiment according to the current invention. The degree of physiological stress or deviation is defined by a vector cross product between the ‘Normal’ vector 100 and the Hemodynamic State Vector 102, and a vector 104 represents the vector cross product. The vector cross product is a product of the length of the ‘Normal’ vector 100, the length of the Hemodynamic State Vector 102 and the sine of the angle between the two vectors. It has a direction of a line perpendicular to the plane that is defined by the original ‘normal’ vector 100 and the hemodynamic state vector 102. It also has an up or down sign relative to the above plane as given by the right hand rule. In general, the longer the length of the vector cross product 104, the more serious the patient's problem is. The length of the vector cross product is just the square root of the dot product of the vector cross product with itself.

Still referring to FIG. 12, as this length of the vector cross product exceeds a set of predetermined thresholds, a corresponding alarm or alert 97 is optionally given to an operator or a clinician. Additional axes involving the oxygen saturation, the end-tidal carbon dioxide and the patient's temperature are alternatively combined in real time to create a multidimensional vector cross product. In another embodiment, other axes could be added as needed or as new modalities of monitoring are developed such as the processed EEG monitor (BIS) to gauge the depth of anesthesia. Obviously, vector spaces in excess of three dimensions are not easily displayed on a screen. But the length of the multidimensional vector cross product is easily displayed and is properly called a continuous Vital Function Scale. Arbitrarily large deviations from the norm automatically disengage the automatic pilot mode of the neuro-cardiovascular control process and alert the clinician to immediately correct the situation before the patient's life is threatened.

The above described displays as illustrated in FIGS. 10, 11 and 12 afford the clinician more time by providing the relevant cardiac data to rectify the problems. As the length of the vector cross product increases, the clinician is at least visually alerted as to the level of deviation from the norm. Furthermore a computer program is implemented to quickly point the clinician's attention to which system or component of the multidimensional vector is a source of the problem so as to save precious seconds and to allow more time for a critical intervention for patient safety. The above displays of FIGS. 10, 11 and 12 also substantially reduce the level of skill needed to recognize the problem. In addition to the clinicians, some technicians who have not had the benefit of a medical school education would quickly be able to visually understand the significance of the information in an intuitive manner. Since the system does not require arcane anatomic image or physiological waveform interpretation skills, the skill level is substantially reduced for responding to the same information. The above described preferred embodiments are likely to be used with a short learning curve by anyone who can read a graph and substantially lower the cost of health care.

All the buttons and display areas as illustrated in FIGS. 10, 11 and 12 are combined in any manner, and the users optionally customize a combination and the location of each button and display area.

Depending on how the hemodynamic state vector moves, various vasoactive agents are brought to bear upon the problem so as to move the patient's hemodynamics back toward the norm. Agents such as phenylephrine, nitroglycerine, nitroprusside, dopamine, dobutamine and esmolol are likely to help to stabilize sick patients undergoing the highly variable stresses of surgery. Vasoactive drug infusions are currently underutilized because not enough patients have the full metal jacket invasive cardiovascular monitoring that is needed to benefit from them. The system according to the present invention increases the wider usage of the easily adjustable vasoactive drugs that are now only routinely used during cardiac surgery.

Yet another aspect of the present invention encompasses a method and device for performing equipotency assays on different formulations of the same anesthetizing agent marketed by different companies, or performing equipotency assays on different agents or classes of agents. A human study population volunteers to be anesthetized. In the case of an intravenous agent of a first formulation, the above described method and/or system is used to deliver a known quantity of the intravenous agent sufficient to depress the subject's neurological activity to some predetermined level, which is generally agreed to represent general anesthesia. The depressed neurological activity is monitored by the BIS, other processed EEG, or alternative anesthetic depth monitoring technology. For example, the predetermined level corresponds to a BIS level of 50-60. The patient is allowed to come to equilibrium, at a pre-determined temperature, oxygen saturation, and end-tidal CO2 level, following the automatic administration of a certain necessary vasoactive agent or combination of agents by the above described method and/or system to place the hemodynamic state vector in some known and well quantified position with respect to Preload, Afterload, Contractility, Stroke Volume, Heartrate, and cardiac Output. The patient's venous blood is then assayed for the concentration of the study drug necessary to achieve the predetermined level of central nervous system depression.

Next, the same procedure is followed with the other manufacturer's formulation (a second formulation) of the same drug, and the same patient is brought to the identical level of neurological depression. The vasoactive agents are administered by the present invention to achieve a hemodynamic state substantially identical to the one using the first formulation. In this way, the equipotency assay is controlled for the several variables that represent the patient's hemodynamic state. The patient's venous blood is once again assayed for anesthetic drug concentration. For a given study population and a particular drug formulation, the assayed venous drug concentrations are averaged, and the standard deviation is calculated. The population averages of the intravenous anesthetic agent venous blood concentrations at equilibrium are then compared, and the difference between them is calculated. The comparison is made between average agent venous blood concentrations at equilibrium. Hemodynamic, temperature, oxygenation, and respiratory parameters are controlled for. This comparison, or difference between average equilibrium agent concentration values for the two study populations is essentially an equipotency assay for multiple formulations of an intravenous anesthetic agent such as Propofol.

The procedure for comparing inhalational agents is similar. The essential difference is that instead of assaying venous blood for drug concentrations, we measure the end-tidal concentration of the anesthetic agent in volumes percent using a mass spectrometer, infra-red absorbance spectroscope or other device all of which are generally ubiquitous in all hospital anesthetizing locations. The measurement is taken for a particular formulation of a particular agent on a population of volunteers. The patient's temperature, oxygen saturation, end-tidal CO2, and hemodynamic parameters are all controlled. This is noted when making the comparison between the mean end-tidal agent concentrations at identical BIS values for different agents.

This method and apparatus is also used to compare and establish equipotency doses between intravenous and inhalational anesthetizing agents. It can be used to create graphs of mean agent concentration as a function of BIS depression level, while controlling temperature, oxygen saturation, end-tidal CO2, and multiple hemodynamic parameters. It can also be used to determine and quantify the synergistic effects of other adjuvant drugs on central nervous system depression commonly used during peni-operatively such as opioids, muscle relaxants, and alpha-2 agonists etc. as a function of the dose of said adjuvant agents, and their concentration on populations while controlling in an automatic feedback controlled fashion for inherent changes in hemodynamics.

We claim:

1. A method of administering a general anesthetic, comprising the steps of:
non-invasively measuring a set of predetermined non-invasive cardiac and neurological parameters from a subject;
converting the non-invasive cardiac parameters into a plurality of invasive cardiac analogues based upon a first set of predetermined conversion equations;
converting the non-invasive neurological parameters into a neurological index based upon a second set of predetermined conversion equations;
administering a general anesthetic based upon neurological index to maintain a desirable level of neurological depression in the subject; and
independently administering a vasoactive agent based upon the converted invasive cardiac analogues to restore cardiovascular homeostasis in the subject.

2. The method of administering a general anesthetic according to claim 1 wherein the subject is a human.

3. The method of administering a general anesthetic according to claim 1 wherein the subject is an animal.

4. The method of administering a general anesthetic according to claim 1 wherein the predetermined non-invasive cardiac parameters include heart rate as denoted by HR, ejection interval as denoted by EI, mean arterial pressure as denoted by MAP and electrical-mechanical interval as denoted by E-M, which is an interval between an electrical event E and a mechanical event M.

5. The method of administering a general anesthetic according to claim 4 wherein the predetermined invasive cardiac analogues include preload as denoted by P, afterload as denoted by A and contractility as denoted by C.

6. The method of administering a general anesthetic according to claim 5 wherein the predetermined conversion equations include
P=k1(EI*MAP*E-M)+c1 or P=k4(DI*MAP*E-M)+c4,
A=k2(MAP*E-M)+c2, and
ln(C)=k3(1/E-M)+c3
where k1, k2, k3, k4, c1, c2, c3 and c4 are empirical proportionality constants.

7. The method of administering a general anesthetic according to claim 6 wherein the M in the E-M is defined as a time when a second derivative with respect to time, M″(t), reaches a maximum value.

8. The method of administering a general anesthetic according to claim 6 wherein the electrical event in determining the E-M is selected from the group consisting of a Q-wave, a R-wave, an S-wave, and an artificial ventricular pacemaker spike.

9. The method of administering a general anesthetic according to claim 6 wherein the electrical event in determining the E-M interval is determined by double differentiating an EKG voltage curve which corresponds to ventricular depolarization, V(t), with respect to time and defining the electrical event as a time when V″(t) reaches a maximum positive value.

10. The method of administering a general anesthetic according to claim 6 wherein the second mechanical event in determining the E-M is selected from the group consisting of arterial blood pressure and flow velocity upstroke.

11. The method of administering a general anesthetic according to claim 5 wherein the predetermined conversion equations include
P=k1′((T-EI)*MAP*E-M)+c1′ or P=k4′(DI*MAP*E-M)+c4′,
A=k2′(MAP*E-M)+c2′, and
ln(C)=k3′(1/E-M)+c3′
where k1′, k2′, k3′, k4′, c1′, c2′, c3′ and c4′ are empirical proportionality constants for a particular one of the subjects and T is a time period of the cardiac cycle for the particular one of the subjects.

12. The method of administering a general anesthetic according to claim 11 wherein the M in the E-M is defined as a time when a second derivative with respect to time, M″(t), reaches a maximum value.

13. The method of administering a general anesthetic according to claim 11 wherein the electrical event in determining the E-M is selected from the group consisting of a Q-wave, a R-wave, an S-wave, and an artificial ventricular pacemaker spike.

14. The method of administering a general anesthetic according to claim 11 wherein the electrical event in determining the E-M interval is determined by double differentiating an EKG voltage curve which corresponds to ventricular depolarization, V(t), with respect to time and defining the electrical event as a time when V″(t) reaches a maximum positive value.

15. The method of administering a general anesthetic according to claim 13 wherein a mechanical event in determining the E-M includes a time of an arterial blood pressure upstroke as denoted by TA and a time of a blood flow velocity upstroke as denoted by TF.

16. The method of administering a general anesthetic according to claim 5 wherein the predetermined conversion equations include
P=k1′((T-EI)*MAP*E-M)+c1′ or P=k4′(DI*MAP*E-M)+c4′,
A=k2′*MAP/[K+exp(1/E-M)]+c2′, and
ln(C)=k3′(1/E-M)+c3′
where k1′, k2′, k3′, k4′, c1′, c2′, c3′, c4′ and K are empirical proportionality constants for a particular one of the subjects and T is a time period of the cardiac cycle for the particular one of the subjects.

17. The method of administering a general anesthetic according to claim 4 wherein the EI is measured by placing a Doppler ultrasound device over the suprasternal notch near the ascending aorta.

18. The method of administering a general anesthetic according to claim 4 wherein the electrical event E in the E-M is determined by electrocardiograph as denoted by EKG.

19. The method of administering a general anesthetic according to claim 4 wherein the E-M is determined by electrocardiograph as denoted by EKG and by placing a Doppler ultrasound device over a major artery.

20. The method of administering a general anesthetic according to claim 4 wherein the E-M is determined by electrocardiograph as denoted by EKG and by placing a fiberoptic device over a major artery.

21. The method of administering a general anesthetic according to claim 5 further comprising the step of displaying the invasive cardiac analogues in three dimensional coordinate space that is defined by a first axis indicative of the P, a second axis indicative of the A and a third axis indicative of the C.

22. The method of administering a general anesthetic according to claim 21 further comprising an additional step of displaying a three dimensional object defining a safe zone indicative of a safe hemodynamic state.

23. The method of administering a general anesthetic according to claim 22 wherein the first axis, the second axis, the third axis and the three dimensional object are each displayed with a predetermined color.

24. The method of administering a general anesthetic according to claim 1 further comprising an additional step of displaying a vector cross product between a first vector indicating an amount of physiologic stress in a current certain situation and a second vector indicating an amount of physiologic stress under a normal condition.

25. The method of administering a general anesthetic according to claim 1 further comprising an additional step of displaying information on vital signs of the subject.

26. The method of administering a general anesthetic according to claim 1 further comprising an additional step of displaying identification information on the subject.

27. The method of administering a general anesthetic according to claim 1 wherein said neurological parameter is measured in EEG and converted into a BIS index.

28. The method of administering a general anesthetic according to claim 1 wherein said administering step for the general anesthetic further comprises additional steps of:
retrieving dosage information for the general anesthetic;
selecting the general anesthetic;
determining a dose of the selected general anesthetic based upon the retrieved dosage information;
generating an anesthetic delivery command including the determined dose and the selected general anesthetic;
delivering the selected general anesthetic to the subject according to the anesthetic delivery command; and
recording information contained in the anesthetic delivery command.

29. The method of administering a general anesthetic according to claim 28 wherein said dosage information includes any combination of physical characteristics of the subject, a dosage and an algorithm for modifying the dosage.

30. The method of administering a general anesthetic according to claim 28 wherein said dosage determining step further comprises additional steps of:
comparing the neurological index to comparison information to generate a comparison result;
generating a warning signal if a critical condition exists in the subject based upon the comparison result; and
displaying the warning signal.

31. The method of administering a general anesthetic according to claim 30 wherein said comparison information includes a pair of threshold values and past ones of the neurological values.

32. The method of administering a general anesthetic according to claim 31 wherein said comparing step compares the neurological index to the threshold values to see if the neurological index exceeds either of the threshold values.

33. The method of administering a general anesthetic according to claim 31 wherein said comparing step compares the neurological index to the past ones of the neurological values to see if the neurological index has changed over a predetermined amount of time.

34. The method of administering a general anesthetic according to claim 30 wherein said delivering step for the general anesthetic is stopped based upon the warning signal without human intervention.

35. The method of administering a general anesthetic according to claim 1 further comprising an additional step of displaying a value of the neurological index.

36. The method of administering a general anesthetic according to claim 1 further comprising an additional step of stopping said administering the general anesthetic.

37. The method of administering a general anesthetic according to claim 36 further comprising an additional step of resuming said administering the general anesthetic.

38. The method of administering a general anesthetic according to claim 1 wherein said administering step for the vasoactive agent further comprises additional steps of:
retrieving dosage information for the vasoactive agent, selecting the vasoactive agent;
determining a dose of the selected vasoactive agent based upon the retrieved dosage information;
generating a vasoactive agent delivery command including the determined dose and the selected vasoactive agent;
delivering the selected vasoactive agent to the subject according to the vasoactive agent delivery command; and
recording information contained in the vasoactive agent delivery command.

39. The method of administering a general anesthetic according to claim 38 wherein said dosage information includes any combination of physical characteristics of the subject, a dosage and an algorithm for modifying the dosage.

40. The method of administering a general anesthetic according to claim 38 wherein said dosage determining step further comprises additional steps of:
comparing each of the converted invasive cardiac analogues to comparison information to generate a comparison result;
generating a warning signal if a critical condition exists in the subject based upon the comparison result; and
displaying the warning signal.

41. The method of administering a general anesthetic according to claim 40 wherein said comparison information includes pairs of threshold values and past ones of the converted invasive cardiac analogues.

42. The method of administering a general anesthetic according to claim 41 wherein said comparing step compares each of the converted invasive cardiac analogues to a corresponding to one of the pairs of the threshold values to see if any of the converted invasive cardiac analogues exceeds either of the corresponding pair of the threshold values.

43. The method of administering a general anesthetic according to claim 41 wherein said comparing step compares each of the converted invasive cardiac analogues to corresponding ones of the past converted invasive cardiac analogues to see if any of the converted invasive cardiac analogues has changed over a predetermined amount of time.

44. The method of administering a general anesthetic according to claim 40 wherein said delivering step for the vasoactive agent is stopped based upon the warning signal without human intervention.

45. The method of administering a general anesthetic according to claim 1 further comprising an additional step of displaying values of the converted invasive cardiac analogues.

46. The method of administering a general anesthetic according to claim 1 further comprising an additional step of stopping said administering the vasoactive agent.

47. The method of administering a general anesthetic according to claim 46 further comprising an additional step of resuming said administering the vasoactive agent.

48. The method of administering a general anesthetic according to claim 1 further comprising additional steps of monitoring respiratory parameters of the subject; and determining if the subject is properly respired based upon the monitored respiratory parameters before the vasoactive agent is considered to be administered based upon the converted invasive cardiac analogues.

49. The method of administering a general anesthetic according to claim 48 wherein said monitored respiratory parameters include a CO₂ wave, a CO₂ level, a pulse oximeter measured value and an oxygen concentration.

50. A system for administering a general anesthetic, comprising:
a non-invasive neurological parameter measuring unit for non-invasively measuring at least a predetermined neurological parameter from the subject;
a neurological parameter conversion unit connected to said non-invasive neurological parameter measuring unit for converting the non-invasive neurological parameter into a neurological index based upon a first set of predetermined conversion equations;
a general anesthetic administering unit connected to said neurological parameter conversion unit for administering a general anesthetic based upon the neurological index to maintain a desirable level of neurological depression in the subject;
a non-invasive cardiac parameter measuring unit for non-invasively measuring a plurality of predetermined non-invasive cardiac parameters from the subject;
a cardiac parameter conversion unit connected to said non-invasive cardiac parameter measuring unit for converting the non-invasive cardiac parameters into a plurality of invasive cardiac analogues based upon a second set of predetermined conversion equations; and
a vasoactive agent administering unit connected to said cardiac parameter conversion unit for administering a vasoactive agent based upon the invasive cardiac analogues to restore cardiovascular homeostasis in the subject.

51. The system for administering a general anesthetic according to claim 50 wherein said non-invasive cardiac parameter measuring unit measures the predetermined non-invasive cardiac parameters from a human.

52. The system for administering a general anesthetic according to claim 50 wherein said non-invasive cardiac parameter measuring unit measures the predetermined non-invasive cardiac parameters from an animal.

53. The system for administering a general anesthetic according to claim 50 wherein said non-invasive cardiac parameter measuring unit further comprises a heart rate monitor for measuring heart rate as denoted by HR, an ultrasonic flow measuring device or electrical impedance measuring device for measuring an ejection interval as denoted by EI and a mechanical event M of an electrical-mechanical interval as denoted by E-M, a blood pressure measuring device for measuring mean arterial pressure as denoted by MAP and an electrocardiogram measuring device for measuring an electrical event E of the electrical-mechanical interval.

54. The system for administering a general anesthetic according to claim 53 wherein said vibration sensing device comprises at least one of a Doppler ultrasound device and a fiber optic device.

55. The system for administering a general anesthetic according to claim 50 wherein said cardiac parameter conversion unit outputs the predetermined invasive cardiac analogues including preload as denoted by P, afterload as denoted by A and contractility as denoted by C.

56. The system for administering a general anesthetic according to claim 55 wherein said cardiac parameter conversion unit determines the P, the A and the C based upon the predetermined conversion equations including
P=k1(EI*MAP*E-M)+c1,
A=k2(MAP*E-M)+c2, and
ln(C)=k3(1/E-M)+c3
where k1, k2, k3, c1, c2 and c3 are empirical proportionality constants.

57. The system for administering a general anesthetic according to claim 55 wherein said cardiac parameter conversion unit determines the P, the A and the C based upon the predetermined conversion equations including
P=k1′((T-EI)*MAP*E-M)+c1′,
A=k2′(MAP*E-M)+c2′, and
ln(C)=k3′(1/E-M)+c3′
where k1′, k2′, k3′, c1′, c2′ and c3′ are empirical proportionality constants for a particular one of the subjects and T is a time period of the cardiac cycle for the particular one of the subjects.

58. The system for administering a general anesthetic according to claim 55 wherein said cardiac parameter conversion unit determines the P, the A and the C based upon the predetermined conversion equations including
P=k1′(DI*MAP*E-M)+c1′,
A=k2′(MAP*E-M)+c2′, and
ln(C)=k3′(1/E-M)+c3′
where k1′, k2′, k3′, c1′, c2′ and c3′ are empirical proportionality constants for a particular one of the subjects, DI is a diastolic filling interval, and T is a time period of the cardiac cycle for the particular one of the subjects.

59. The system for administering a general anesthetic according to claim 57 wherein said cardiac parameter conversion unit obtains the mechanical event M in the E-M by determining a time when a second derivative with respect to time, M″(t) reaches a maximum value.

60. The system for administering a general anesthetic according to claim 59 wherein said non-invasive cardiac parameter measuring unit measures an electrical event in determining the E-M, the electrical event being selected from the group consisting of a Q-wave as denoted by Q, a R-wave as denoted by R, a S-wave as denoted by S and an artificial ventricular pacemaker spike.

61. The system for administering a general anesthetic according to claim 59 wherein said non-invasive cardiac parameter measuring unit measures an electrical event in determining the E-M interval by double differentiating an EKG voltage curve which corresponds to ventricular depolarization, V(t), with respect to time and defining the electrical event as a time when V″(t) reaches a maximum positive value.

62. The system for administering a general anesthetic according to claim 60 wherein said non-invasive cardiac parameter measuring unit measures the mechanical event in determining the E-M, the second mechanical event including a time of an arterial blood pressure upstroke as denoted by TA and a time of a flow velocity upstroke as denoted by TF.

63. The system for administering a general anesthetic according to claim 55 wherein said cardiac parameter conversion unit determines the P, the A and the C based upon the predetermined conversion equations including
P=k1′((T-EI)*MAP*E-M)+c1′ or P=k4′(DI*MAP*E-M)+c4′,
A=k2′*MAP/[K+exp(1/E-M)]+c2′, and
ln(C)=k3′(1/E-M)+c3′
where k1′, k2′, k3′, k4′, c1′, c2′, c3′, c4′ and K are empirical proportionality constants for a particular one of the subjects and T is a time period of the cardiac cycle for the particular one of the subjects.

64. The system for administering a general anesthetic according to claim 55 further comprising a display unit connected to said cardiac parameter conversion unit for displaying the invasive cardiac analogues in three dimensional coordinate space that is defined by a first axis indicative of the P, a second axis indicative of the A and a third axis indicative of the C.

65. The system for administering a general anesthetic according to claim 64 wherein said display unit additionally displays a three dimensional object defining a safe zone indicative of a safe hemodynamic state.

66. The system for administering a general anesthetic according to claim 65 wherein said display unit displays the first axis, the second axis, the third axis and the safe zone respectively in a predetermined color.

67. The system for administering a general anesthetic according to claim 65 wherein said display unit additionally displays a vector cross product indicative of an amount of physiologic stress.

68. The system for administering a general anesthetic according to claim 50 further comprising a display unit connected to said cardiac parameter conversion unit for displaying information on vital signs of the subject.

69. The system for administering a general anesthetic according to claim 50 further comprising a display unit for displaying identification information on the subject.

70. The system for administering a general anesthetic according to claim 50 wherein said non-invasive neurological parameter measuring unit measures the neurological parameter in EEG and said neurological parameter conversion unit converts the EEG into a BIS index.

71. The system for administering a general anesthetic according to claim 50 wherein said general anesthetic administering unit further comprises:
a neurological control unit for retrieving dosage information for the general anesthetic, selecting the general anesthetic, determining a dose of the selected general anesthetic based upon the retrieved dosage information and generating an anesthetic delivery command including the determined dose and the selected general anesthetic; and
an anesthetic agent delivery unit connected to said neurological control unit for delivering the selected general anesthetic to the subject according to the anesthetic delivery command and recording information in the anesthetic delivery command in a database.

72. The system for administering a general anesthetic according to claim 71 wherein said dosage information includes any combination of physical characteristics of the subject, a dosage and an algorithm for modifying the dosage.

73. The system for administering a general anesthetic according to claim 71 wherein said neurological control unit compares the neurological index to comparison information to generate a comparison result and generates a warning signal if a critical condition exists in the subject based upon the comparison result.

74. The system for administering a general anesthetic according to claim 73 further comprising a display unit connected to said neurological control unit for displaying the warning signal.

75. The system for administering a general anesthetic according to claim 73 wherein the comparison information includes a pair of threshold values and past ones of the neurological values.

76. The system for administering a general anesthetic according to claim 75 wherein said neurological control unit compares the neurological index to the threshold values to see if the neurological index exceeds either of the threshold values.

77. The system for administering a general anesthetic according to claim 75 wherein said neurological control unit compares the neurological index to the past ones of the neurological values to see if the neurological index has changed over a predetermined amount of time.

78. The system for administering a general anesthetic according to claim 73 wherein said anesthetic agent delivery unit is disengaged based upon the warning signal without human intervention.

79. The system for administering a general anesthetic according to claim 50 wherein said general anesthetic administering unit further comprises:
a neurological control unit connected to said non-invasive neurological parameter measuring unit for selecting a first general anesthetic and determining a first dose for the general anesthetic based upon the neurological index value;
a central monitoring and delivering control unit connected to said neurological control unit for retrieving dosage information for the general anesthetic, selecting a second general anesthetic, independently determining a second dose of the selected general anesthetic based upon the retrieved dosage information, resolving between the first general anesthetic and the second general anesthetic, resolving between the first dose and the second dose, and generating an anesthetic delivery command including the resolved dose and the resolved general anesthetic; and
an anesthetic agent delivery unit connected to said neurological control unit and said central monitoring and delivering control unit for delivering the general anesthetic to the subject according to the anesthetic delivery command and recording information contained in the anesthetic delivery command in a database.

80. The system for administering a general anesthetic according to claim 79 wherein the dosage information includes any combination of physical characteristics of the subject, a dosage and an algorithm for modifying the dosage.

81. The system for administering a general anesthetic according to claim 79 wherein said central monitoring and delivering control unit compares the neurological index to comparison information to generate a comparison result and generates a warning signal if a critical condition exists in the subject based upon the comparison result.

82. The system for administering a general anesthetic according to claim 81 further comprising a display unit connected to said central monitoring and delivering control unit for displaying the warning signal.

83. The system for administering a general anesthetic according to claim 79 wherein the comparison information includes a pair of threshold values and past ones of the neurological values.

84. The system for administering a general anesthetic according to claim 83 wherein said central monitoring and delivering control unit compares the neurological index to the threshold values to see if the neurological index exceeds either of the threshold values.

85. The system for administering a general anesthetic according to claim 83 wherein said central monitoring and delivering control unit compares the neurological index to the past ones of the neurological values to see if the neurological index has changed over a predetermined amount of time.

86. The system for administering a general anesthetic according to claim 81 wherein said anesthetic agent delivery unit is disengaged based upon the warning signal without human intervention.

87. The system for administering a general anesthetic according to claim 50 further comprising a display unit connected to said neurological parameter conversion unit for displaying a value of the neurological index.

88. The system for administering a general anesthetic according to claim 71 wherein said general anesthetic administering unit stops said anesthetic agent delivery unit.

89. The system for administering a general anesthetic according to claim 88 wherein said general anesthetic administering unit resumes said anesthetic agent delivery unit.

90. The system for administering a general anesthetic according to claim 50 wherein said vasoactive agent administering unit further comprises:
a cardiovascular control unit for retrieving dosage information for the vasoactive agent, selecting the vasoactive agent, determining a dose of the selected vasoactive agent based upon the retrieved dosage information and generating a vasoactive agent delivery command including the determined dose and the selected vasoactive agent; and
a vasoactive agent delivery unit connected to said cardiovascular control unit for delivering the selected vasoactive agent to the subject according to the vasoactive agent delivery command and recording information in the vasoactive agent delivery command in a database.

91. The system for administering a general anesthetic according to claim 90 wherein said dosage information includes any combination of physical characteristics of the subject, a dosage and an algorithm for modifying the dosage.

92. The system for administering a general anesthetic according to claim 90 wherein said cardiovascular control unit compares each of the converted invasive cardiac analogues to comparison information to generate a comparison result and generates a warning signal if a critical condition exists in the subject based upon the comparison result.

93. The system for administering a general anesthetic according to claim 92 further comprising a display unit connected to said cardiovascular control unit for displaying the warning signal.

94. The system for administering a general anesthetic according to claim 92 wherein the comparison information includes pairs of threshold values and past ones of the converted invasive cardiac analogues.

95. The system for administering a general anesthetic according to claim 94 wherein said cardiovascular control unit compares each of the converted invasive cardiac analogues to one of the corresponding pairs of the threshold values to see if any of the converted invasive cardiac analogues exceeds either of the corresponding pair of the threshold values.

96. The system for administering a general anesthetic according to claim 94 wherein said cardiovascular control unit compares each of the converted invasive cardiac analogues to corresponding ones of the past converted invasive cardiac analogues to see if any of the converted invasive cardiac analogues has changed over a predetermined amount of time.

97. The system for administering a general anesthetic according to claim 92 wherein said vasoactive agent delivery unit is disengaged based upon the warning signal without human intervention.

98. The system for administering a general anesthetic according to claim 50 further comprising a display unit connected to said cardiac parameter conversion unit for displaying values of the converted invasive cardiac analogues.

99. The system for administering a general anesthetic according to claim 50 wherein said vasoactive agent administering unit further comprises:
a cardiovascular control unit connected to said non-invasive cardiac parameter measuring unit for selecting a first vasoactive agent and determining a first dose for the vasoactive agent based upon the non-invasive cardiac parameters;
a central monitoring and delivering control unit connected to said cardiovascular control unit for retrieving dosage information for the vasoactive agent, selecting a second vasoactive agent, independently determining a second dose of the selected vasoactive agent based upon the retrieved dosage information, resolving between the first vasoactive agent and the second vasoactive agent, resolving between the first dose and the second dose, and generating a vasoactive agent delivery command including the resolved dose and the resolved vasoactive agent; and
a vasoactive agent delivery unit connected to said cardiovascular control unit and said central monitoring and delivering control unit for delivering the vasoactive agent to the subject according to the vasoactive agent delivery command and recording information contained in the vasoactive agent delivery command in a database.

100. The system for administering a general anesthetic according to claim 99 wherein the dosage information includes any combination of physical characteristics of the subject, a dosage and an algorithm for modifying the dosage.

101. The system for administering a general anesthetic according to claim 99 wherein said central monitoring and delivering control unit compares the invasive cardiac analogues to comparison information to generate a comparison result and generates a warning signal if a critical condition exists in the subject based upon the comparison result.

102. The system for administering a general anesthetic according to claim 101 further comprising a display unit connected to said central monitoring and delivering control unit for displaying the warning signal.

103. The system for administering a general anesthetic according to claim 101 wherein the comparison information includes pairs of threshold values and past ones of the invasive cardiac analogues.

104. The system for administering a general anesthetic according to claim 103 wherein said central monitoring and delivering control unit compares each of the invasive cardiac analogues to a corresponding pair of the threshold values to see if the invasive cardiac analogue exceeds either of the threshold values.

105. The system for administering a general anesthetic according to claim 103 wherein said central monitoring and delivering control unit compares each of the invasive cardiac analogues to a corresponding one of the past ones of the cardiac values to see if the invasive cardiac analogues have changed over a predetermined amount of time.

106. The system for administering a general anesthetic according to claim 101 wherein said vasoactive agent delivery unit is disengaged based upon the warning signal without human intervention.

107. The system for administering a general anesthetic according to claim 50 wherein said vasoactive agent administering unit stops said vasoactive agent delivery unit.

108. The system for administering a general anesthetic according to claim 107 wherein said vasoactive agent administering unit resumes said vasoactive agent delivery unit.

109. The system for administering a general anesthetic according to claim 50 further comprising a respiratory monitoring unit connected to said vasoactive agent administering unit for monitoring respiratory parameters of the subject and for determining if the subject is properly respired based upon the monitored respiratory parameters before said vasoactive agent administering unit considers the vasoactive agent to be administered based upon the converted invasive cardiac analogues.

110. The system for administering a general anesthetic according to claim 109 wherein the monitored respiratory parameters include a CO₂ wave, a CO₂ level, a pulse oximeter measured value and an oxygen concentration.

111. A method of performing equipotency assays on anesthetizing agents, comprising the steps of:
a) anesthetizing a subject with a first anesthetizing agent to a certain predetermined level;
b) maintaining a level of neurological depression by a predetermined procedure;
c) monitoring a cardiovascular state of the patient;
d) achieving cardiovascular equilibrium in the cardiovascular state;
e) assaying a concentration of the first anesthetizing agent in blood of the patient; and
f) repeating the above steps a) through e) with a second anesthetizing agent with the same subject to determine whether or not the first anesthetizing agent and second anesthetizing agent achieve a substantially identical hemodynamic state.

112. The method of performing equipotency assays on anesthetizing agents according to claim 111 wherein the second anesthetizing agent is a different formulation of the first anesthetizing agent.

113. The method of performing equipotency assays on anesthetizing agents according to claim 111 wherein the second anesthetizing agent is a different drug from the first anesthetizing agent.

114. The method of performing equipotency assays on anesthetizing agents according to claim 111 wherein said steps c) and d) of monitoring the cardiovascular state and achieving cardiovascular equilibrium further comprise steps of:
non-invasively measuring a set of predetermined non-invasive cardiac parameters from the subject;
converting the non-invasive cardiac parameters into a plurality of invasive cardiac analogues based upon a first set of predetermined conversion equations; and
administering a vasoactive agent based upon the converted invasive cardiac analogues to restore cardiovascular equilibrium in the subject.

115. The method of performing equipotency assays on anesthetizing agents according to claim 111 wherein said step b) of maintaining the level of neurological depression further comprises steps of:
non-invasively measuring a set of predetermined non-invasive neurological parameters from the subject;
converting the non-invasive neurological parameters into a neurological index based upon a second set of predetermined conversion equations; and
administering the anesthetizing agent based upon the neurological index to maintain a desirable level of neurological depression in the subject.

116. The method of performing equipotency assays on anesthetizing agents according to claim 111 wherein said step c) of monitoring the cardiovascular state is indicated by a hemodynamic state vector with respect to Preload, Contractility and Afterload.

117. A system for performing equipotency assays on anesthetizing agents, comprising:
a general anesthetic administering unit for anesthetizing a subject separately with a first anesthetizing agent and a second anesthetizing agent to a certain predetermined level;
a non-invasive neurological parameter measuring unit connected to said general anesthetic administering unit for non-invasively measuring at least a predetermined neurological parameter from the subject, wherein said general anesthetic administering unit maintains a level of neurological depression by a predetermined procedure;
a non-invasive cardiac parameter measuring unit for non-invasively measuring cardiac parameters and monitoring a cardiovascular state of the patient;
a vasoactive agent administering unit connected to said non-invasive cardiac parameter measuring unit for achieving cardiovascular equilibrium; and
a central monitoring and delivering unit for recording respective concentrations of the first anesthetizing agent and the second anesthetizing agent in blood of the patient and for determining whether or not the first anesthetizing agent and second anesthetizing agent achieve a substantially identical hemodynamic state.

118. The system for performing equipotency assays on anesthetizing agents according to claim 117 wherein the second anesthetizing agent is a different formulation of the first anesthetizing agent.

119. The system for performing equipotency assays on anesthetizing agents according to claim 117 wherein the second anesthetizing agent is a different drug from the first anesthetizing agent.

120. The system for performing equipotency assays on anesthetizing agents according to claim 117 wherein said general anesthetic administering unit further comprises a cardiac parameter conversion unit connected to said non-invasive cardiac parameter measuring unit for converting the non-invasive cardiac parameters into a plurality of invasive cardiac analogues based upon a first set of predetermined conversion equations, wherein said vasoactive agent administering unit administers a vasoactive agent based upon the converted invasive cardiac analogues to restore cardiovascular equilibrium in the subject.

121. The system for performing equipotency assays on anesthetizing agents according to claim 117 wherein said general anesthetic administering unit further comprise a neurological parameter conversion unit connected to said non-invasive neurological parameter measuring unit for converting the non-invasive neurological parameters into a neurological index based upon a second set of predetermined conversion equations, wherein said general anesthetic administering unit administers the anesthetizing agent based upon the neurological index to maintain a desirable level of neurological depression in the subject.

122. The system for performing equipotency assays on anesthetizing agents according to claim 117 wherein said non-invasive cardiac parameter measuring unit monitors the cardiovascular state of the patient as indicated by a hemodynamic state vector with respect to Preload, Contractility and Afterload.

123. A method of simulating cardiovascular and neurological conditions of a patient under a general anesthetic, comprising the steps of:
inputting first data representing characteristics of a patient;
inputting second data representing an anesthetic agent;
simulating neurological conditions and cardiovascular conditions in response the second data based upon the first data;
displaying information representing at least some of the simulated neurological conditions and the simulated cardiovascular conditions;
audio-visually warning any undesirable condition; and
allowing human intervention if the undesirable condition exists.

124. The method of simulating cardiovascular and neurological conditions according to claim 123 wherein the information optionally includes description of underlying physiological events involved in the simulated cardiovascular conditions.

125. The method of simulating cardiovascular and neurological conditions according to claim 123 wherein the simulated cardiovascular conditions include a pulse, an EKG, an E-M interval, an EI or DI interval and a blood pressure or a mean arterial pressure.

126. The method of simulating cardiovascular and neurological conditions according to claim 123 wherein the simulated neurological conditions include an EEG.

127. The method of simulating cardiovascular and neurological conditions according to claim 123 wherein said inputting the second data is interactively performed.

128. The method of simulating cardiovascular and neurological conditions according to claim 123 further comprising additional steps of:
inputting third data representing a vasoactive agent to restore cardiovascular equilibrium in the patient; and
further simulating the cardiovascular conditions in response the third data based upon the first data.

129. The method of simulating cardiovascular and neurological conditions according to claim 123 wherein the cardiovascular conditions of the patient are indicated by a hemodynamic state vector with respect to Preload, Contractility and Afterload.

130. A system for simulating cardiovascular and neurological conditions of a patient under a general anesthetic, comprising:
a patient characteristic database for storing first data representing characteristics of a patient and second data representing an anesthetic agent;
a simulation module connected to said patient characteristic database for inputting the first data and the second data, said simulation module simulating neurological conditions and cardiovascular, conditions in response the second data based upon the first data; and
a display unit connected to said simulation module for displaying information representing at least some of the simulated neurological conditions and the simulated cardiovascular conditions, said display unit audio-visually warning any undesirable condition and allowing to receive an input indicative of human intervention if the undesirable condition exists.

131. The system for simulating cardiovascular and neurological conditions according to claim 130 wherein the information optionally includes description of underlying physiological events involved in the simulated cardiovascular conditions.

132. The system for simulating cardiovascular and neurological conditions according to claim 130 wherein the simulated cardiovascular conditions include a pulse, an EKG, an E-M interval, an EI or DI interval and a blood pressure or a mean arterial pressure.

133. The system for simulating cardiovascular and neurological conditions according to claim 130 wherein the simulated neurological conditions include an EEG.

134. The system for simulating cardiovascular and neurological conditions according to claim 130 wherein said inputting of the second data is interactively performed.

135. The system for simulating cardiovascular and neurological conditions according to claim 130 wherein said patient characteristic database stores third data representing a vasoactive agent for restoring cardiovascular equilibrium in the patient, said simulation module inputting third data and further simulating the cardiovascular conditions in response to the third data based upon the first data.

136. The system for simulating cardiovascular and neurological conditions according to claim 130 wherein the cardiovascular conditions of the patient are indicated by a hemodynamic state vector with respect to Preload, Contractility and Afterload.

137. The method of administering a general anesthetic according to claim 5 wherein the predetermined conversion equations include
SV=k6′EIe^{(1/E-M)}+c6′
where k6′and c6′are empirical proportionality constants.

138. The method of administering a general anesthetic according to claim 4 wherein the EI and DI are measured by placing a Doppler ultrasound device on the chest over the left ventricle or by measuring a transthoracic electrical impedance waveform.
